(19) United States
(12) Patent Application Publication
(10) Pub. No.: US 2011/0190372 A1
(43) Pub. Date: Aug. 4, 2011

(54) COMPOSITIONS AND METHODS FOR TREATING INFLAMMATORY DISORDERS

(75) Inventors: Marjana Tomic-Canic, Hillsdale, NJ (US); Harold Brem, Bronx, NY (US)

(73) Assignee: NEW YORK UNIVERSITY, NEW YORK, NY (US)

(21) Appl. No.: 12/853,065

(22) Filed: Aug. 9, 2010

Related U.S. Application Data
(60) Provisional application No. 61/232,282, filed on Aug. 7, 2009.

(51) Int. Cl. A61K 31/7052; A61P 29/00

(52) U.S. Cl. 514/44 A

(57) ABSTRACT

Compositions and methods for antagonizing miRNAs that are overexpressed in chronic, non-healing wounds, as compared to healthy tissue, are disclosed. The miRNA antagonists are oligonucleotides that hybridize to selected pre-miRNA or mature miRNAs and prevent the miRNAs from binding to and downregulating their target mRNAs. Methods of using the miRNA antagonists to treat inflammatory disorders, including to promote healing of chronic, non-healing wounds and acute wounds are provided.

### CROSS REFERENCE TO RELATED APPLICATIONS

This application claims benefit of U.S. Provisional Application No. 61/232,282, filed Aug. 7, 2009, which is hereby incorporated herein by reference in its entirety.

### GOVERNMENT SUPPORT

The United States government has certain rights in this invention by virtue of National Institutes of Health grant No. UL1RR024996 to M. Tomic-Canic.

### FIELD OF THE INVENTION

The invention is generally related to compositions and methods for promoting wound healing.

### BACKGROUND OF THE INVENTION

It is estimated that each year more than 8 million people in the United States develop chronic non-healing wounds, including pressure, venous, diabetic ulcers and burns (Harsha, et al., J. Mol. Med., 86(8):961-9 (2008)). The prevalence of diabetes worldwide was estimated to be 2.8% in 2000 and estimated to increase 4.4% in 2030 (Wild, et al., Diabetes Care, 27(5):1047-53 (2004); Narayan, et al., JAMA, 290(14):1884-90 (2003); Fong, et al., Diabetes Care, 27(Suppl 1):S84-7 (2004); Turner, et al., Prog. Drug Res., 51:33-94 (1998)). Among persons diagnosed with diabetes mellitus, the lifetime risk of developing a foot ulcer may be as high as 25% (Singh, et al., JAMA, 293(2):217-28 (2005); Layery, et al., Diabetes Care, 26(5):1435-8 (2003)). Lower extremity disease, including peripheral arterial disease, peripheral neuropathy, foot ulceration, or lower extremity amputation, is twice as common in diabetic persons and affects 30% of diabetic persons who are older than 40 years. Digit and limb amputations are 15 times more common in people with diabetes than in those without the disease and foot ulcers precede approximately 84% of non-traumatic amputations in persons with diabetes. Foot ulcers cause substantial emotional, physical, productivity, and financial losses. In the United States alone, more than 82,000 limb amputations are performed in patients with diabetes each year. These amputations are associated with disability, drastic decline in quality of life, and an alarming mortality rate of 39%-69% over 5 years.

Chronic wounds are characterized by physiological impairments manifested by delays in healing, resulting in severe morbidity. A number of physiological factors contribute to wound healing deficiencies in individuals with diabetes. These include decreased growth factor production (Cianfarani, et M., Am. J. Pathol., 169(4):1167-82 (2006); Tanaka, et al., Biol. Pharm. Bull., 19(9):1141-8 (1996); Werner, et al., J. Invest. Dermatol., 103(4):469-73 (1994); Blakytny, et al., J. Pathol., 190(5):589-94 (2000)) impaired macrophage function Cianfarani, et al., Am. J. Pathol., 169(4):1167-82 (2006); Tanaka, et al., Biol. Pharm. Bull., 19(9):1141-8 (1996); Okumura, et al., Biol. Pharm. Bull., 19(4):530-5 (1996)) reduced collagen accumulation, delayed epidermal barrier function, decreased quality and quantity of granulation tissue (Keswani, et al., Wound Repair Regen., 12(5):497-504 (2004); Galiano, et al., Am. J. Pathol., 164(6):1935-47 (2004)) keratinocyte and fibroblast migration and proliferation (Spravchikov, et M., Diabetes, 50(7):1627-35 (2001); Puricelli, et al., J. Clin. Endocrinol. Metab., 91(9):3507-14 (2006); Lerman, et al., Am. J. Pathol., 162(1):303-12 (2003)), vascular complications (Veves, et al., Diabetes, 47(3):457-63 (1998); Schramm, et al., Int. J. Low. Extrem, Wounds, 5(3):149-59 (2006); Goldin, et al., Circulation, 114(6):597-605 (2006)), fewer number of epidermal nerves (Gibran, et al., J. Surg. Res., 108(1):122-8 (2002); Spenny, et al., Wound Repair Regen., 10(5):295-301 (2002)), reduced bone healing and angiogenic deficiencies (Tanaka, et al., Biol. Pharm. Bull., 19(9):1141-8 (1996); Okumura, et al., Biol. Pharm. Bull., 19(4):530-5 (1996)), vascular disturbances and impairment of inflammatory response (Galkowska, et al., J. Surg. Res., 134(2):252-8 (2006); Veves, et al., Diabetes, 47(3):457-63 (1998); Schramm, et al., Int. J. Low. Extrem, Wounds, 5(3):149-59 (2006)). Diabetic foot ulcer (DFU) also has an impaired balance between the accumulation of ECM components, and their remodeling by matrix metalloproteinases (MMPs). Clinical trials of exogenously administered growth factors such as TGFβ1, KGF and EGF to human chronic ulcers have achieved very limited efficacy and failed to obtain FDA approval, despite early promising animal studies (Mustoe, et al., Science, 237(4820):1333-6 (1987); Sporn and Roberts, J. Clin. Invest., 92(6):2565-6 (1993)).

Currently there are only two products available on the market approved by the FDA for treatment of chronic wounds: platelet derived growth factor (PDGF-BB), (Smiell, Am. J. Surg., 176(2A Suppl):68S-73S (1998); Wieman, et al., Diabetes Care, 21(5):822-7 (1998); Wieman, Am. J. Surg, 176(2A Suppl):74S-79S (1998); LeGrand, Am. J. Surg., 176(2A Suppl):48S-54S (1998)) and human skin equivalent (Muhart, et al., Arch. Dermatol., 135(8):913-8 (1999); Brem, et al., Arch. Surg., 135(6):627-34 (2000); Sibbald, J. Cutan. Med. Surg., 3 Suppl 1:S1-24-8 (1998)). However, there are several problems with these therapies, such as neither has demonstrated efficacy in venous ulcers (VUs) or ischemic DFUs; and both have a minimal failure rate of 44% in well-vascularized limbs that are properly off-loaded. Although this is better than the failure rate of standard therapies (i.e., off-loading and saline dressing), the number of amputations and non-healed VUs and DFUs remains excessive. Nonetheless, these therapies have been ground-breaking in demonstrating that local therapy is clinically effective in the treatment of chronic wounds.

Previous studies have identified pathogenic markers in patients' biopsies from the non-healing edge of venous ulcers that correlate with delayed wound healing (Stojadinovic, et al., Am. J. Pathol., 167(1):59-69 (2005); Brem, et al., Mol. Med., 13(1-2):30-9 (2007)). Microarray analyses of these biopsies have shown that the transcriptional profiles of epithelial cells from the two wound locations, the non-healing edge and adjacent non-ulcerated skin, are biologically distinct (Brem, et al., Mol. Med., 13(1-2):30-9 (2007)). Data using venous ulcers indicates suppression of a large number of transcription-related genes in patients with chronic ulcers.

Therefore, it is object of the invention to provide compositions and methods to inhibit or reduce the suppression of transcription-related genes in wounds.

It is another object of the invention to provide compositions and methods to promote wound healing.

It is yet another object of the invention to provide compositions and methods for treating other inflammatory conditions.

### SUMMARY OF THE INVENTION

Compositions for antagonizing miRNAs that are overexpressed in chronic, non-healing wounds, as compared to healthy tissue, are disclosed. The miRNA antagonists are oligonucleotides that hybridize to selected pre-miRNA or mature miRNAs and prevent the miRNAs from binding to and downregulating their target mRNAs. The miRNA antagonists include single-stranded, double-stranded, and partially double-stranded or hairpin structured oligonucleotides that include nucleotides sequences that are complementary to a selected miRNA.

In some embodiments, the miRNA antagonists contain nucleotide sequences that are complementary to a portion of a miRNA listed in Table 1 below. In preferred embodiments, the miRNA antagonist is sufficiently complementary to a portion of the miRNA or pre-miRNA sequence of a human miRNA such as miR-21, miR-590-5p, miR-15a, miR-15b, miR-16-1, miR-16-2, miR-195, miR-424, miR-497, miR-17-5p, miR-20a, miR-20b, miR-93.mr, miR-106a, miR-106b, miR-519.d, miR-106a, miR-106b, miR-302a, miR-302b, miR-302c, miR-302d, miR-302e, miR-302f, miR-103-1, miR-103-2, miR-103-1-as, miR-103-2-as, miR-107, miR-130a, miR-130b, miR-301a, miR-301b, miR-27a, miR-27b, miR-143, miR-146a, miR-146b and miR-203.

The miRNA antagonists can be oligomers or polymers of RNA or DNA, and can contain modifications to their nucleobases, sugar groups, phosphate groups, or covalent internucleoside linkages. Preferred modifications include those that increase the stability of the miRNA antagonists or enhance cellular uptake of the miRNA antagonists. In one embodiment, the miRNA antagonists are antagomirs, which have 2′-O-methylation of the sugars, a phosphorothioate backbone and a terminal cholesterol moiety.

Methods of using the miRNA antagonists to inhibit or reduce the expression or biological activity of miRNAs overexpressed in chronic non-healing wounds are provided. In one embodiment, the miRNA antagonists are administered to an individual in an effective amount to promote wound healing. Wounds that may be treated include chronic, non-healing wounds, such as diabetic ulcers, arterial ulcers, venous ulcers, pressure ulcers and burns. In another embodiment, the miRNA antagonists are used to promote healing of acute wounds, such as those caused by acute injury or surgery. The wound to be treated may be in any epithelial tissue, including skin, mouth tissue, gingival and corneal epithelium. In other embodiments, the miRNA antagonists are used to treat other inflammatory conditions including, but not limited to, arthritis, osteoarthritis, rheumatoid arthritis, asthma, vasculitis, inflammatory bowel disease, pelvic inflammatory disease, inflammatory skin disorders, multiple sclerosis, osteoporosis, tendonitis, allergic disorders, sepsis, atherosclerosis, and systematic lupus erythematosus.

The miRNA antagonists can be topically or subcutaneously administered at or adjacent to the site of a wound to be treated. They may be formulated into sustained release formulations and may be incorporated into wound dressings or wound inserts. For the treatment of other inflammatory conditions, miRNA antagonists can be administered by other routes including, but not limited to, orally, by inhalation (nasal or pulmonary), intravenously, intraperitoneally, intramuscularly, sublingually, or rectally. The miRNA antagonists can be used in combination with other therapies for treating wounds, including, but not limited to, anti-microbial agents, pain relievers, anti-inflammatory agents and growth factors.

### BRIEF DESCRIPTION OF THE DRAWINGS

FIG. 1A is a bar graph showing the relative expression of the miRNAs miR-20a, miR-16, miR-21 and miR-203 in normal skin and in skin from non-healing edges of chronic, non-healing wounds. Data are expressed as the relative expression of the miRNA normalized to U48 RNA expression levels.

FIG. 1B is a bar graph showing the relative expression of the miRNAs miR-106a, and miR-130a in normal skin and in skin from non-healing edges of chronic, non-healing wounds. Data are expressed as the relative expression of the miRNA normalized to U48 RNA expression levels.

FIG. 2A is a bar graph showing the relative expression of miR-20a in normal skin and in skin from non-healing edges of diabetic foot ulcers (DFUs) and venous ulcers (VUs). Data are expressed as the relative expression of the miRNA normalized to U48 RNA expression levels.

FIG. 2B is a bar graph showing the relative expression of miR-21 in normal skin and in skin from non-healing edges of diabetic foot ulcers (DFUs) and venous ulcers (VUs). Data are expressed as the relative expression of the miRNA normalized to U48 RNA expression levels.

FIG. 3 is bar graph showing the relative expression levels of TGFβRII mRNA in normal skin and in skin from non-healing edges of VUs. Data were obtained using quantitative RT-PCR, and are expressed as the relative expression of the miRNA normalized to U48 RNA expression levels.

FIG. 4 is a schematic showing alignment of sequences within miR-21 and miR-130a with target sites for these miRNAs in the 3′ untranslated region of LepR mRNA.

FIG. 5 is a bar graph showing relative luciferase reporter activity in primary human keratinocytes co-transfected with a luciferase reporter construct under control of the LepR 3′ UTR sequence in either the sense (LepR 3′ UTR-S) or antisense (LepR 3′ UTR-AS) orientations and either an empty pSilencer vector (control), or pSilencer expressing miR-21 or miR-130a. Data are expressed as relative luciferase activity.

FIG. 6A is a bar graph showing the migration of primary cultured human fibroblasts derived from the non-healing edge of chronic wounds as a function of time. Migration of cells was measured as the percent wound coverage after inducing a “scratch” in the cell monolayer in culture. The percent wound coverage was measured at 0 hours (empty bar), 4 hours (gray bar), 8 hours (hatched bar) and 24 hours (black bar).

FIG. 6B is a bar graph showing the migration of primary cultured human fibroblasts derived from the non-healing edge of chronic wounds as a function of time. Cells were cultured in the presence of vascular endothelial growth factor (VEGF). Migration of cells was measured as the percent wound coverage after inducing a “scratch” in the cell monolayer in culture. The percent wound coverage was measured at 0 hours (empty bar), 4 hours (gray bar), 8 hours (hatched bar) and 24 hours (black bar).

FIG. 7 is a bar graph showing relative expression of Dicer in healthy skin (empty bar) and venus ulcers (VU) (black bar). Data are expressed as the relative expression of the Dicer normalized to HPRT1 expression levels.

FIG. 8 is a bar graph showing relative expression of miR-21 (first two bars) and miR-27b (last two bars) in control porcine wounds (empty bars) and porcine wounds infected with biofilm-forming Pseudomonas (black bars). Data are expressed as the relative expression of the miRNA normalized to GAPDH mRNA expression levels.

### DETAILED DESCRIPTION OF THE INVENTION

### I. Compositions

MicroRNAs (miRNAs) are a class of 18-24 nucleotide (“nt”) non-coding RNAs (ncRNAs) that exist in a variety of organisms, including mammals, that can suppress the expression of protein-coding genes by targeting RNA-induced silencing complex (RISC) to cognate messenger RNA targets (Ambros, Nature, 431(7006):350-5 (2004); Bartel, Cell, 116(2):281-97 (2004)). miRNAs are generated by sequential processing of long hairpin precursors of about 70 nucleotides (pre-miRNA) which are derived from primary transcripts (pri-miRNA) through sequential cleavage by two key RNase III proteins, Drosha and Dicer (Kim, Nat. Rev. Mol. Cell. Mal., 6(5):376-85 (2005)). Many microRNAs can be encoded in intergenic regions, hosted within introns of pre-mRNAs or within ncRNA genes. Many miRNAs also tend to be clustered and transcribed as polycistrons and often have similar spatial temporal expression patterns.

In mammals, miRNAs use a seed sequence of 6-8 nt to associate with 3′ untranslated regions (3′ UTRs) of mRNAs and inhibit their expression. These regulators of gene expression are capable of defining and altering cell fate. Recent estimations suggest the existence of 500-1000 miRNAs per genome and that a large proportion of human protein-coding genes are under the regulation of one or more miRNAs (Aravin and Tuschl, FEBS Lett., 579:5830-5840 (2005); Lewis, et al., Cell, 120:15-20 (2005); Selbach, Nature, 455(7209):58-63 (2008)).

miRNAs participate in the regulation of the large variety of cellular processes including developmental timing, differentiation, apoptosis, cell proliferation, organ development, and metabolism, and the vast majority of miRNAs show tissue or developmental stage-specific expression (Lagos-Quintana, et al., Curr. Biol., 12:735-739 (2002); Lim, et al., Nature, 433:769-773 (2005); Wienholds, et al., Science, 309:310-311 (2005); Landgraf, Cell, 129(7):1401-14 (2007)).

Evidence suggests a significant role for miRNAs in a wide range of physiologic functions in mammals, including insulin secretion, heart, skeletal muscle and brain development (Kloosterman, et al., Dev. Cell, 11:441-50 (2006); Krutzfeldt, et al., Cell. Metab., 4:9-12 (2006)). Importantly, aberrant expression or activity of miRNAs can lead to disease; in particular, miRNAs are often aberrantly expressed in cancer (Croce and Calin, Cell, 122:6-7 (2005); Kloosterman and Plasterk, Dev. Cell, 11:441-450 (2006); Pillai, et al., Trends Cell Biol., 17:118-126 (2007); Voorhoeve and Agami, Cell, 124:1169-1181 (2006)). Thus, miRNAs represent important targets for potential therapeutic and diagnostic agents (Czech, N. Engl. J. Med., 354:1194-1195 (2006)).

The importance of miRNAs in epidermal development and adult skin stem cell maintenance has been described recently (Yi, Nat. Genet., 38:356-362 (2006); Andl, Curr. Biol., 16, 1041-1049 (2006); Yi, Nature, 452:225-229 (2008)). When miRNAs are ablated in skin epithelium by conditionally targeting Dicer1, barrier function of the epithelium is compromised and hairs fail to invaginate, indicating the functional importance of these small RNAs in skin development. Specific miRNAs expressed in developing mouse epidermis and hair follicles were identified (Yi, Nat. Genet., 38:356-362 (2006); Andl, Curr. Biol., 16, 1041-1049 (2006)). The most abundant epidermal miRNA, miR-203, was shown to promote epidermal differentiation by restricting sternness and inducing cell-cycle exit by directly repressing the expression of p63 (Yi, Nature, 452:225-229 (2008)). In adult epidermis, miRNA-203 is not expressed in proliferating basal keratinocytes but is detectable in the upper layers, and is rapidly upregulated when primary keratinocytes are induced to differentiate by calcium (Lena, Cell Death Differ., 15:1187-1195 (2008)). A recent study has shown involvement of specific miRNAs in pathology of psoriasis, and miR-203 was also found to be upregulated in psoriatic plaques (Sonkoly, PLoS ONE, 2(7):e610 (2007)).

It has now been discovered that microRNAs (miRNAs) are overexpressed in chronic non-healing wounds as compared to healthy skin. More specifically, it has been discovered that miRNAs are overexpressed in epithelial cells at non-healing edges of a non-healing wounds as compared to epithelial cells from adjacent healthy skin. The examples below demonstrate that miRNAs overexpressed in chronic wounds target mRNAs that encode proteins that function in wound healing and inhibit acute wound healing in organ culture wound models. It is believed that induction of these miRNAs contributes to attenuation of growth factor signaling and general transcriptional attenuation that occurs in chronic non-healing wounds.

A. miRNA Antagonists

Compositions that function as miRNA antagonists to down-regulate expression and function of miRNAs that are overexpressed in chronic wounds are disclosed. The miRNA antagonists form a duplex with target miRNAs, which prevents the miRNA from binding to its target mRNA. This results in increased translation of the mRNA that is targeted by the miRNA, including mRNAs that encode for proteins that function in wound healing.

The disclosed miRNA antagonists are single-stranded, double stranded, partially double stranded or hairpin structured oligonucleotides that include a nucleotide sequence sufficiently complementary to hybridize to a selected miRNA or pre-miRNA target sequence. As used herein, the term “partially double stranded” refers to double stranded structures that contain less nucleotides than the complementary strand. In general, partially double stranded oligonucleotides will have less than 75% double stranded structure, preferably less than 50%, and more preferably less than 25%, 20% or 15% double stranded structure.

An miRNA or pre-miRNA can be 18-100 nucleotides in length, and more preferably from 18-80 nucleotides in length. Mature miRNAs can have a length of 19-30 nucleotides, preferably 21-25 nucleotides, particularly 21, 22, 23, 24, or 25 nucleotides. MicroRNA precursors typically have a length of about 70-100 nucleotides and have a hairpin conformation.

Given the sequence of an miRNA or a pre-miRNA, an miRNA antagonist that is sufficiently complementary to a portion of the miRNA or a pre-miRNA can be designed according to the rules of Watson and Crick base pairing. As used herein, the term “sufficiently complementary” means that two sequences are sufficiently complementary such that a duplex can be formed between them under physiologic conditions. An miRNA antagonist sequence that is sufficiently complementary to an miRNA or pre-miRNA target sequence can be 70%, 80%, 90%, or more identical to the miRNA or pre-miRNA sequence. In one embodiment, the miRNA antagonist contains no more than 1, 2 or 3 nucleotides that are not complementary to the miRNA or pre-miRNA target sequence. In a preferred embodiment, the miRNA antagonist is 100% complementary to an miRNA or pre-miRNA target sequence.

In some embodiments, the miRNA antagonist is sufficiently complementary to a portion of the miRNA or pre-miRNA sequence of a human miRNA listed in Table 1 below. Sequences for these miRNAs are available publicly through the miRBase registry (Griffiths-Jones, et al., Nucleic Acids Res., 36(Database Issue):D154-D158 (2008); Griffiths-Jones, et al., Nucleic Acids Res., 36(Database Issue):D140-D144 (2008); Griffiths-Jones, et al., Nucleic Acids Res., 36(Database Issue):D109-D111 (2008)), and miRBase Accession Nos. are indicated in Table 1.

In preferred embodiments, the miRNA antagonist is sufficiently complementary to a portion of the miRNA or pre-miRNA sequence of a human miRNA selected from miR-21, miR-590-5p, miR-15a, miR-15b, miR-16-1, miR-16-2, miR-195, miR-424, miR-497, miR-17-5p, miR-20a, miR-20b, miR-93.mr, miR-106a, miR-106b, miR-519.d, miR-106a, miR-106b, miR-302a, miR-302b, miR-302c, miR-302d, miR-302e, miR-302f, miR-103-1, miR-103-2, miR-103-1-as, miR-103-2-as, miR-107, miR-130a, miR-130b, miR-301a, miR-301b, miR-27a, miR-27b, miR-143, miR-146a, miR-146b and miR-203.

The miRNA antagonist can have a region that is at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to a portion of the miRNA or pre-miRNA sequence of a human miRNA selected from miR-21, miR-590-5p, miR-15a, miR-15b, miR-16-1, miR-16-2, miR-195, miR-424, miR-497, miR-17-5p, miR-20a, miR-20b, miR-93.mr, miR-106a, miR-106b, miR-519.d, miR-106a, miR-106b, miR-302a, miR-302b, miR-302c, miR-302d, miR-302e, miR-302f, miR-103-1, miR-103-2, miR-103-1-as, miR-103-2-as, miR-107, miR-130a, miR-130b, miR-301a, miR-301b, miR-27a, miR-27b, miR-143, miR-146a, miR-146b and miR-203.

Useful miRNA antagonists include oligonucleotides have at least 6, 7, 8, 9, 10, 11, 12, 13, 14, 15, 16, 17, 18, 19, 20 or more contiguous nucleotides substantially complementary to an endogenous miRNA or pre-miRNA that is overexpressed in epithelial cells at non-healing edges of a non-healing wounds as compared to epithelial cells from adjacent healthy skin. The disclosed miRNA antagonists preferably include a nucleotide sequence sufficiently complementary to hybridize to an miRNA target sequence of about 12 to 25 nucleotides, preferably about 15 to 23 nucleotides.

In some embodiments, there will be nucleotide mismatches in the region of complementarity. In a preferred embodiment, the region of complementarily will have no more than 1, 2, 3, 4, or 5 mismatches.

In some embodiments, the miRNA antagonist is “exactly complementary” to a human miRNA selected from miR-21, miR-590-5p, miR-15a, miR-15b, miR-16-1, miR-16-2, miR-195, miR-424, miR-497, miR-17-5p, miR-20a, miR-20b, miR-93.mr, miR-106a, miR-106b, miR-519.d, miR-106a, miR-106b, miR-302a, miR-302b, miR-302c, miR-302d, miR-302e, miR-302f, miR-103-1, miR-103-2, miR-103-1-as, miR-103-2-as, miR-107, miR-130a, miR-130b, miR-301a, miR-301b, miR-27a, miR-27b, miR-143, miR-146a, miR-146b and miR-203. Thus, in one embodiment, the miRNA antagonist can anneal to the miRNA to form a hybrid made exclusively of Watson-Crick base pairs in the region of exact complementarity. Thus, in some embodiments, the miRNA antagonist specifically discriminates a single-nucleotide difference. In this case, the miRNA antagonist only inhibits miRNA activity if exact complementarity is found in the region of the single-nucleotide difference.

In one embodiment, the miRNA antagonists are oligomers or polymers of ribonucleic acid (RNA) or deoxyribonucleic acid (DNA) or modifications thereof. miRNA antagonists include oligonucleotides that contain naturally-occurring nucleobases, sugars and covalent internucleoside (backbone) linkages.

1. Bases

The miRNA antagonists can contain modified bases. Adenine, guanine, cytosine and uracil are the most common bases found in RNA. These bases can be modified or replaced to provide RNAs having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine). Alternatively, substituted or modified analogs of any of the above bases can be used. Examples include, but are not limited to, 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases.

The disclosed miRNA antagonists can be modified to enhanced resistance to nucleases. Thus, the disclosed miRNA antagonists can be an oligomer that includes nucleotide modification that stabilized it against nucleolytic degradation. The oligomer can be a totalmer, mixmer, gapmer, tailmer, headmer or blockmer. A “totalmer” is a single stranded oligonucleotide that only comprises non-naturally occurring nucleotides. The term “gapmer” refers to an oligonucleotide composed of modified nucleic acid segments flanking at least 5 naturally occurring nucleotides (i.e., unmodified nucleic acids). The term “blockmer” refers to a central modified nucleic acid segment flanked by nucleic acid segments of at least 5 naturally occurring nucleotides. The term “tailmer” refers to an oligonucleotide having at least 5 naturally occurring nucleotides at the 5′-end followed by a modified nucleic acid segment at the 3′-end. The term “headmer” refers to oligonucleotide having a modified nucleic acid segment at the 5′-end followed by at least 5 naturally occurring nucleotides at the 3′-end. The term “mixmer” refers to oligonucleotide which comprise both naturally and non-naturally occurring nucleotides. However, unlike gapmers, tailmers, headmers and blockmers, there is no contiguous sequence of more than 5 naturally occurring nucleotides, such as DNA units.

Modified nucleic acids and nucleotide surrogates can include one or more of: (i) replacement of one or both of the non-linking phosphate oxygens and/or of one or more of the linking phosphate oxygens; (ii) replacement of a constituent of the ribose sugar, e.g., of the 2′ hydroxyl on the ribose sugar, or wholesale replacement of the ribose sugar with a structure other than ribose; (iii) wholesale replacement of the phosphate moiety with “dephospho” linkers; (iv) modification or replacement of a naturally occurring base; (v) replacement or modification of the ribose-phosphate backbone; or (vi) modification of the 3′ end or 5′ end of the RNA, e.g., removal, modification or replacement of a terminal phosphate group or conjugation of a moiety, such as a fluorescently labeled moiety, to either the 3′ or 5′ end of RNA.

2. The Sugar Group

The miRNA antagonists can contain modified sugar groups. For example, the 2′ hydroxyl group (OH) can be modified or replaced with a number of different “oxy” or “deoxy” substitutents.

Examples of “oxy”-2′ hydroxyl group modifications include alkoxy or aryloxy, “locked” nucleic acids (LNA) in which the 2′ hydroxyl is connected, for example, by a methylene bridge or ethylene bridge to the 4′ carbon of the same ribose sugar; amino, O-AMINE and aminoalkoxy. Oligonucleotides containing only methoxyethyl groups (MOE) exhibit nuclease stabilities comparable to those modified with the robust phosphorothioate modification.

“Deoxy” modifications include hydrogen, halo, amino, cyano; mercapto, alkyl-thio-alkyl, thioalkoxy, and alkyl, cycloalkyl, aryl, alkenyl and alkynyl, which may be optionally substituted. Preferred substitutents are 2′-methoxyethyl, 2′-OCH3, 2′-O-allyl, 2′-C-allyl, and 2′-fluoro.

The sugar group can also contain one or more carbons that possess the opposite stereochemical configuration than that of the corresponding carbon in ribose. Thus, a modified RNA can include nucleotides containing e.g., arabinose, as the sugar.

Also included are “abasic” sugars, which lack a nucleobase at C-1′. These abasic sugars can also be further contain modifications at one or more of the constituent sugar atoms.

To maximize nuclease resistance, the 2′ modifications can be used in combination with one or more phosphate linker modifications (e.g., phosphorothioate). The so-called “chimeric” oligonucleotides are those that contain two or more different modifications.

3. The Phosphate Group

The disclosed miRNA antagonists can contain modified phosphate groups. The phosphate group is a negatively charged species. The charge is distributed equally over the two non-linking oxygen atoms. However, the phosphate group can be modified by replacing one of the oxygens with a different substitutent. One result of this modification to RNA phosphate backbones can be increased resistance of the oligoribonucleotide to nucleolytic breakdown.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphorus center in the phosphorodithioates is achiral which precludes the formation of oligoribonucleotides diastereomers. Diastereomer formation can result in a preparation in which the individual diastereomers exhibit varying resistance to nucleases. Further, the hybridization affinity of RNA containing chiral phosphate groups can be lower relative to the corresponding unmodified RNA species.

The phosphate group can be replaced by non-phosphorus containing connectors. Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. Preferred replacements include the methylenecarbonylamino and methylenemethylimino groups.

Oligonucleotide-mimicking scaffolds can also be constructed wherein the phosphate linker and ribose sugar are replaced by nuclease resistant nucleoside or nucleotide surrogates. Examples include the mophilino, cyclobutyl, pyrrolidine and peptide nucleic acid (PNA) nucleoside surrogates. A preferred surrogate is a PNA surrogate.

4. Terminal Modifications

The disclosed miRNA antagonists can also be modified at their 3′ and/or 5′ ends. Terminal modifications can be added for a number of reasons, including to modulate activity, to modulate resistance to degradation, or to modulate uptake of the miRNA antagonists by cells. Modifications can include modification or replacement of an entire terminal phosphate or of one or more of the atoms of the phosphate group. For example, the 3′ and 5′ ends of an oligonucleotide can be conjugated to other functional molecular entities such as labeling moieties or protecting groups. The functional molecular entities can be attached to the sugar through a phosphate group and/or a spacer. The terminal atom of the spacer can connect to or replace the linking atom of the phosphate group or the C-3′ or C-5′ O, N, S or C group of the sugar. Alternatively, the spacer can connect to or replace the terminal atom of a nucleotide surrogate. Other examples of terminal modifications include dyes, intercalating agents, cross-linkers, porphyrins, polycyclic aromatic hydrocarbons, artificial endonucleases, lipophilic carriers and peptide conjugates.

5. Antagomirs

In some embodiments, the miRNA antagonists are antagomirs. Antagomirs are a specific class of miRNA antagonists that are described, for example, in US2007/0213292 to Stoffel et al. Antagomirs are RNA-like oligonucleotides that contain various modifications for RNase protection and pharmacologic properties such as enhanced tissue and cellular uptake. Antagomirs differ from normal RNA by having complete 2′-O-methylation of sugar, phosphorothioate backbone and a cholesterol-moiety at 3′-end.

Antagomirs can include a phosphorothioate at least the first, second, or third internucleotide linkage at the 5′ or 3′ end of the nucleotide sequence. In one embodiment, antagomirs contain six phosphorothioate backbone modifications; two phosphorothioates are located at the 5′-end and four at the 3′-end. Phosphorothioate modifications provide protection against RNase activity and their lipophilicity contributes to enhanced tissue uptake.

Examples of antagomirs and other miRNA inhibitors are described in WO2009/020771, WO2008/091703, WO2008/046911, WO2008/074328, WO2007/090073, WO2007/027775, WO2007/027894, WO2007/021896, WO2006/093526, WO2006/112872, WO2007/112753, WO2007/112754, WO2005/023986, or WO2005/013901, all of which are hereby incorporated by reference.

Custom designed Anti-miR™ molecules are commercially available from Applied Biosystems. Thus, in some embodiments, the antagomir is an Ambian® Anti-miR™ inhibitor. These molecules are chemically modified and optimized single-stranded nucleic acids designed to specifically inhibit naturally occurring mature miRNA molecules in cells.

Custom designed Dharmacon Meridian™ microRNA Hairpin Inhibitors are also commercially available from Thermo Scientific. These inhibitors include chemical modifications and secondary structure motifs. For example, Vermeulen et al. reports in US2006/0223777 the identification of secondary structural elements that enhance the potency of these molecules. Specifically, incorporation of highly structured, double-stranded flanking regions around the reverse complement core significantly increases inhibitor function and allows for multi-miRNA inhibition at subnanomolar concentrations. Other such improvements in antagomir design are contemplated for use in the disclosed methods.

B. Carriers

1. Nucleic Acid Delivery Vehicles

miRNA antagonists can be introduced into the skin or other external tissues with agents that can facilitate uptake into epithelial cells using a variety of techniques that are available in the art. For example, miRNA antagonists can be introduced into cells using mechanical methods, such as microinjection, liposome-mediated transfection, iontophoresis, or calcium phosphate precipitation. In some embodiments, the disclosed miRNA antagonists are formulated in the form of a controlled release formulation or sustained release formulation administered topically or injected directly into the skin adjacent to or within the area to be treated (intradermally or subcutaneously).

In other embodiments, the disclosed miRNA antagonists can be expressed within cells using vector systems with appropriate eukaryotic promoters.

i. Condensing Agents and Liposomes

In some embodiments, miRNA antagonists can be combined with a condensing agent to form a nucleic acid delivery vehicle. Suitable polycations include, for example, polylysine, polyarginine, polyornithine, protamine, spermine, spermidine, and putrescine. Many suitable methods for making linkages between condensing agents and nucleic acids are known in the art.

In other embodiments, miRNA antagonists can be associated with a liposome to form a nucleic acid delivery vehicle. Liposomes are small, lipid vesicles comprised of an aqueous compartment enclosed by a lipid bilayer, typically spherical or slightly elongated structures several hundred angstroms in diameter. Under appropriate conditions, a liposome can fuse with the plasma membrane of a cell or with the membrane of an endocytic vesicle within a cell that has internalized the liposome, thereby releasing its contents into the cytoplasm. Prior to interaction with the surface of a cell, however, the liposome membrane acts as a relatively impermeable barrier that sequesters and protects its contents, for example, from degradative enzymes. Additionally, because a liposome is a synthetic structure, specially designed liposomes can be produced that incorporate desirable features.

Liposomal preparations include cationic (positively charged), anionic (negatively charged) and neutral preparations. Cationic liposomes have been shown to mediate intracellular delivery of plasmid DNA, mRNA, and purified transcription factors, in functional form. Cationic liposomes are readily available. For example, N[1-2,3-dioleyloxy)propyl]-N,N,N-triethylammonium (DOTMA) liposomes are available under the trademark Lipofectin™, (GIBCO BRL, Grand Island, N.Y.), Transfectace (DDAB/DOPE) and DOTAP/DOPE (Boerhinger). Other cationic liposomes can be prepared from readily available materials using techniques available in the art.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), and dioleoylphoshatidyl ethanolamine (DOPE). These materials can also be mixed with the DOTMA and DOTAP starting materials in appropriate ratios. Methods for making liposomes using these materials are well known in the art.

The liposomes can comprise multilamellar vesicles (MLVs), small unilamellar vesicles (SUVs), or large unilamellar vesicles (LUVs). The various liposome-nucleic acid complexes are prepared using methods known in the art.

In addition, lipoproteins can be included with a nucleic acid for delivery to a cell. Examples of such lipoproteins include chylomicrons, HDL, IDL, LDL, and VLDL. Mutants, fragments, or fusions of these proteins can also be used. Modifications of naturally occurring lipoproteins can also be used, such as acetylated LDL. These lipoproteins can target the delivery of nucleic acids to cells expressing lipoprotein receptors. In some embodiments, if lipoproteins are included with a nucleic acid, no other targeting ligand is included in the composition. Receptor-mediated targeted delivery of miRNA antagonists to specific tissues can also be used.

ii. Controlled Release Formulations

Controlled or sustained release can be achieved by the addition of time-release additives, such as polymeric structures, matrices, that are available in the art. An insert, transdermal patch, bandage or article used to deliver the disclosed miRNA antagonists can comprise a mixture or coating of polymers that provide release of the active agents at a constant rate over a prolonged period of time.

In some embodiments, the article, transdermal patch, bandage or insert comprises water-soluble pore forming agents, such as polyethylene glycol (PEG) that can be mixed with water insoluble polymers to increase the durability of the insert and to prolong the release of the active ingredients. Such a water-soluble pore-forming agent can be polyethylene glycol, polypropylene glycol, a mixture or polymer of sugars (lactose, sucrose, dextrose, etc.), salts, poloxamers, hydroxypropylcellulose, polyvinyl alcohol and other water-soluble food grade and other excipients.

The inserts, articles, transdermal patches and bandages may also comprise a water insoluble polymer. Examples of such polymers are ethylcellulose, acrylic resins, co-polymer of methacrylic acid and acrylic acid ethyl ester, polylactic acid, PLGA, polyurethane, polyethylene vinyl acetate copolymer, polystyrene-butadiene copolymer and silicone rubber, or mixtures thereof.

These rate controlling polymers can be applied using a continuous coating film during the process of spraying and drying with active agents. The rate controlling film prepared with such a polymer is stable during implantation. The film should have enough strength to withstand tear and inner osmotic pressure, and have the stability not to swell or hydrate during the implantation life. In one embodiment, the coating formulation is used to coat pellets comprising the active ingredients that are compressed to form a solid, biodegradable insert.

Alternatively, miRNA antagonists can be delivered using a sustained release device. Either non-biodegradable or biodegradable matrices can be used for delivery of nucleic acids, although biodegradable matrices are preferred. These may be natural or synthetic polymers, although synthetic polymers are preferred due to the better characterization of degradation and release profiles. The polymer is selected based on the period over which release is desired, generally in the range of at least two to six weeks, although longer periods may be desirable. In some cases linear release may be most useful, although in others a pulse release or “bulk release” may provide more effective results. The polymer may be in the form of a hydrogel (typically absorbing up to about 90% by weight of water), and can optionally be crosslinked with multivalent ions or polymers.

Oligonucleotides can be delivered partially by diffusion but mainly by degradation of the polymeric system. In this case, biodegradable polymers, bioerodible hydrogels, and protein delivery systems are particularly preferred. Examples of non-biodegradable polymers include ethylene vinyl acetate, poly(meth)acrylic acid, polyamides, copolymers and mixtures thereof. Examples of biodegradable polymers include synthetic polymers such as hydroxyacid polymers, for example, polymers of lactic acid and glycolic acid, polyanhydrides, poly(ortho)esters, polyurethanes, poly(butic acid), poly(valeric acid), and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

In one embodiment, the polymeric matrix is in the form of microparticles or nanoparticles. Microparticles can be in the form of microspheres, where the miRNA antagonist is dispersed within a solid polymeric matrix, or microcapsules, where the core is of a different material than the polymeric shell, and the miRNA antagonist is dispersed or suspended in the core, which may be liquid or solid in nature. Unless specifically defined herein, microparticles, microspheres, microcapsules, nanoparticles, nanospheres, and nanocapsules are used interchangeably.

Alternatively, the polymer may be cast as a thin slab or film, ranging from nanometers to four centimeters, a powder produced by grinding or other standard techniques, or even a gel such as a hydrogel. The matrices can be formed by solvent evaporation, spray drying, solvent extraction and other methods known to those skilled in the art.

In another embodiment, sustained release matrices are formed using fibrin. Fibrin-based biomaterial preparations can be used as provisional growth matrices for cells important in tissue repair during wound healing in vivo. The release of growth factor from fibrin-based biomaterials was demonstrated by Wong, et al., Thromb Haemost., 89(3):573-82 (2003). Growth factor was incorporated into the fibrin biomaterials prior to formation of the clots. Clotting resulted in sustained release of growth factor causing angiogenic activity.

Another embodiment provides miRNA antagonists incorporated in a conventional hydrophobic polymer matrix, e.g. of a polylactide, which is made more accessible for water by introducing a hydrophilic unit, e.g. of polyethyleneglycol, polyvinylalcohol, dextran or polymethacrylamide. The hydrophilic contribution to the amphipathic polymer is given by all the ethylene oxide groups in case of a polyethylene glycol unit, by the free hydroxyl groups in the case of a polyvinylalcohol unit or of a dextran unit, and by the amide groups in the case of a polymethyacrylamide unit.

iii. Vectors

In another embodiment, single-stranded miRNA antagonists can be expressed from transcription units within cells using eukaryotic promoters in appropriate DNA/RNA vectors. Suitable vectors include, but are not limited to, DNA plasmids and viral vectors. miRNA antagonist-expressing viral vectors can be constructed based on, but not limited to, adeno-associated virus, retrovirus, adenovirus, lentivirus, or alphavirus. In another embodiment, pol III based constructs are used to express nucleic acid molecules of the invention (U.S. Pat. Nos. 5,902,880 and 6,146,886). Viral vectors capable of producing either persistent or transient expression of miRNA antagonists in cells can be used.

2. Topical Carriers

The disclosed miRNA antagonists can be administered using a syringe, bandage, transdermal patch, insert, or syringe-like applicator, as a liquid, spray, aerosol, ointment, foam, cream, gel, paste, or powder/talc or other solid.

The miRNA antagonists may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents include water and physiologically acceptable buffered saline solutions such as phosphate buffered saline solutions with a substantially neutral pH. Additives may be mixed in with the formulation for maximum or desired efficacy of the delivery system or for the comfort of the patient. Such additives include, for example, lubricants, plasticizing agents, preservatives, gel formers, film formers, cream formers, disintegrating agents, coatings, binders, vehicles, coloring agents, odor controlling agents, humectants, viscosity controlling agents, pH-adjusting agents, and similar agents.

In a preferred embodiment, the compositions contain sufficient amounts of at least one pH buffering agent to ensure that the composition has a final pH of about 3 to about 11, preferably between 6 and 8, most preferably at or near the pH of the skin. Suitable pH modifying agents include, but are not limited to, sodium hydroxide, citric acid, hydrochloric acid, acetic acid, phosphoric acid, succinic acid, sodium hydroxide, potassium hydroxide, ammonium hydroxide, magnesium oxide, calcium carbonate, magnesium carbonate, magnesium aluminum silicates, hydroxyapatite, malic acid, potassium citrate, sodium citrate, sodium phosphate, lactic acid, gluconic acid, tartaric acid, 1,2,3,4-butane tetracarboxylic acid, fumaric acid, diethanolamine, monoethanolamine, sodium carbonate, sodium bicarbonate, triethanolamine, and combinations thereof.

Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetypyridinium chloride, chlorobutanol, phenol, phenylethyl alcohol, thimerosal, and combinations thereof.

The percent by weight of the active agents present in a formulation will depend on various factors, but generally will be from about 0.01% to about 98% of the total weight of the formulation, and typically about 0.1 to about 90% by weight, more typically less than 50%, most typically in the range of 0.5 to 10%.

Reference is also made to the following examples which demonstrate the dose response curves for the formulations applied to appropriate animal models.

i. Emulsions, Ointments and Creams

The compositions can be formulated as emulsions for topical application. An emulsion contains one liquid distributed the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

Suitable surfactants include, but are not limited to, anionic surfactants, non-ionic surfactants, cationic surfactants, and amphoteric surfactants. Examples of anionic surfactants include, but are not limited to, ammonium lauryl sulfate, sodium lauryl sulfate, ammonium laureth sulfate, sodium laureth sulfate, alkyl glyceryl ether sulfonate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium and ammonium salts of coconut alkyl triethylene glycol ether sulfate; tallow alkyl triethylene glycol ether sulfate, tallow alkyl hexaoxyethylene sulfate, disodium N-octadecylsulfosuccinnate, disodium lauryl sulfosuccinate, diammonium lauryl sulfosuccinate, tetrasodium N-(1,2-dicarboxyethyl)-N-octadecylsulfosuccinnate, diamyl ester of sodium sulfosuccinic acid, dihexyl ester of sodium sulfosuccinic acid, dioctyl esters of sodium sulfosuccinic acid, docusate sodium, and combinations thereof.

Examples of nonionic surfactants include, but are not limited to, polyoxyethylene fatty acid esters, sorbitan esters, cetyl octanoate, cocamide DEA, cocamide MEA, cocamido propyl dimethyl amine oxide, coconut fatty acid diethanol amide, coconut fatty acid monoethanol amide, diglyceryl diisostearate, diglyceryl monoisostearate, diglyceryl monolaurate, diglyceryl monooleate, ethylene glycol distearate, ethylene glycol monostearate, ethoxylated castor oil, glyceryl monoisostearate, glyceryl monolaurate, glyceryl monomyristate, glyceryl monooleate, glyceryl monostearate, glyceryl tricaprylate/caprate, glyceryl triisostearate, glyceryl trioleate, glycol distearate, glycol monostearate, isooctyl stearate, lauramide DEA, lauric acid diethanol amide, lauric acid monoethanol amide, lauric/myristic acid diethanol amide, lauryl dimethyl amine oxide, lauryl/myristyl amide DEA, lauryl/myristyl dimethyl amine oxide, methyl gluceth, methyl glucose sesquistearate, oleamide DEA, PEG-distearate, polyoxyethylene butyl ether, polyoxyethylene cetyl ether, polyoxyethylene lauryl amine, polyoxyethylene lauryl ester, polyoxyethylene lauryl ether, polyoxyethylene nonylphenyl ether, polyoxyethylene octyl ether, polyoxyethylene octylphenyl ether, polyoxyethylene oleyl amine, polyoxyethyelen oleyl cetyl ether, polyoxyethylene oleyl ester, polyoxyethylene oleyl ether, polyoxyethylene stearyl amine, polyoxyethylene stearyl ester, polyoxyethylene stearyl ether, polyoxyethylene tallow amine, polyoxyethylene tridecyl ether, propylene glycol monostearate, sorbitan monolaurate, sorbitan monooleate, sorbitan monopalmitate, sorbitan monostearate, sorbitan sesquioleate, sorbitan trioleate, stearamide DEA, stearic acid diethanol amide, stearic acid monoethanol amide, laureth-4, and combinations thereof.

Examples of amphoteric surfactants include, but are not limited to, sodium N-dodecyl-γ-alanine, sodium N-lauryl-γ-iminodipropionate, myristoamphoacetate, lauryl betaine, lauryl sulfobetaine, sodium 3-dodecyl-aminopropionate, sodium 3-dodecylaminopropane sulfonate, sodium lauroamphoacetate, cocodimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and combinations thereof.

Examples of cationic surfactants include, but are not limited to, behenyl trimethyl ammonium chloride, bis(acyloxyethyl)hydroxyethyl methyl ammonium methosulfate, cetrimonium bromide, cetrimonium chloride, cetyl trimethyl ammonium chloride, cocamido propylamine oxide, distearyl dimethyl ammonium chloride, ditallowedimonium chloride, guar hydroxypropyltrimonium chloride, lauralkonium chloride, lauryl dimethylamine oxide, lauryl dimethylbenzyl ammonium chloride, lauryl polyoxyethylene dimethylamine oxide, lauryl trimethyl ammonium chloride, lautrimonium chloride, methyl-1-oleyl amide ethyl-2-oleyl imidazolinium methyl sulfate, picolin benzyl ammonium chloride, polyquaternium, stearalkonium chloride, sterayl dimethylbenzyl ammonium chloride, stearyl trimethyl ammonium chloride, trimethylglycine, and combinations thereof.

Suitable suspending agents include, but are not limited to, alginic acid, bentonite, carbomer, carboxymethylcellulose and salts thereof, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol, triglycerides, methylcellulose, polyoxyethylene fatty acid esters, polyvinylpyrrolidone, propylene glycol alginate, sodium alginate, sorbitan fatty acid esters, tragacanth, and combinations thereof.

Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

Suitable antioxidants include, but are not limited to, butylated hydroxytoluene, alpha tocopherol, ascorbic acid, fumaric acid, malic acid, butylated hydroxyanisole, propyl gallate, sodium ascorbate, sodium metabisulfite, ascorbyl palmitate, ascorbyl acetate, ascorbyl phosphate, Vitamin A, folic acid, flavons or flavonoids, histidine, glycine, tyrosine, tryptophan, carotenoids, carotenes, alpha-Carotene, beta-Carotene, uric acid, pharmaceutically acceptable salts thereof, derivatives thereof, and combinations thereof.

Suitable chelating agents include, but are not limited to, EDTA, disodium edetate, trans-1,2-diaminocyclohexane-N,N,N,N′-tetraaceticacid monohydrate, N,N-bis(2-hydroxyethyl)glycine, 1,3-diamino-2-hydroxypropane-N,N,N′,N′-te-traacetic acid, 1,3-diaminopropane-N,N,N,N′-tetraacetic acid, ethylenediamine-N,N′-diacetic acid, ethylenediamine-N,N′-dipropionic acid, ethylenediamine-N,N′-bis(methylenephosphonic acid), N-(2-hydroxyethyl)ethylenediamine-N,N,N′,N′-triacetic acid, ethylenediamine-N,N,N′,N′-tetrakis(methylenephosphonic acid), O,O′-bis(2-aminoethyl)ethyleneglycol-N,N,N′,N′-tetraacetic acid, N,N-bis(2-hydroxybenzyl)ethylenediamine-N,N-diacetic acid, 1,6-hexamethylenediamine-N,N,N,N′-tetraacetic acid, N-(2-hydroxyethyl)iminodiacetic acid, iminodiacetic acid, 1,2-diaminopropane-N,N,N′,N′-tetraacetic acid, nitrilotriacetic acid, nitrilotripropionic acid, nitrilotris(methylenephosphoric acid), 7,19,30-trioxa-1,4,10,13,16,22,27,33-octaazabicyclo[11,11,11] pentatriacontane hexahydrobromide, triethylenetetramine-N,N,N′,N″,N′″,N′″-hexaacetic acid, and combinations thereof.

Suitable emollients include, but are not limited to, myristyl lactate, isopropyl palmitate, light liquid paraffin, cetearyl alcohol, lanolin, lanolin derivatives, mineral oil, petrolatum, cetyl esters wax, cholesterol, glycerol, glycerol monostearate, isopropyl myristate, lecithin, and combinations thereof.

Suitable humectants include, but are not limited to, glycerin, butylene glycol, propylene glycol, sorbitol, triacetin, and combinations thereof.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays are conveniently delivered from pressurized packs, for example, via a specially shaped closure. Oil-In-Water emulsions can also be utilized in the compositions, patches, bandages and articles. These systems are semisolid emulsions, micro-emulsions, or foam emulsion systems. Usually such a system has a “creamy white” appearance. Typically, the internal oil phase is in the range in percentage composition of about 10% to about 40% oil by weight and the external phase may contain 80% or more water. The oleaginous phase may contain, but is not limited to, long-chain alcohols (cetyl, stearyl), long-chain esters (myristates, palmitates, stearates), long-chain acids (palmitic, stearic), vegetable and animal oils and assorted waxes. These can be made with anionic, cationic, nonionic or amphoteric surfactants, or with combinations especially of the nonionic surfactants.

ii. Insert or Implants

In some embodiments, the active ingredients can be formulated with oleaginous bases or ointments to form a semisolid composition with a desired shape. For example, the composition can be shaped for easy application to, or insertion into, a wound, ulcer, puncture wound or surgical site. This class of formulations includes the active ingredients and semisolids. In addition to the active ingredients, these semisolid compositions can contain dissolved and/or suspended bactericidal agents, preservatives and/or a buffer system. The petrolatum component in many of these bases can be any paraffin ranging in viscosity from mineral oil employing incorporated isobutylene, colloidal silica, or stearate salts to paraffin waxes. White and yellow petrolatums are examples of such systems. Bases of this class can be made by incorporating high-melting waxes into a fluid mineral oil via fusion or by incorporation of polyethylene into mineral oil at elevated temperature. Polysiloxanes (also known as silicones) are suitable for use in these bases and typically have a viscosity in the range of about 0.5 to lo6 centistokes. The organic entities attached to the polysiloxane are preferably lower molecular weight hydrocarbon moieties having from 1 to 8 carbons each, such as lower alkyl, lower alkenyl, phenyl and alkyl substituted phenyl, and phenyl(lower)alkyl, such as benzyl. In such a moiety, each lower alkyl or alkenyl group preferably has 1 to 3 carbons inclusive, such as in a dimethylsiloxane polymer.

Absorption bases can be used with such an oleaginous system. In addition to the active ingredients, additional ingredients with the capacity to emulsify a significant quantity of water are employed. Water-in-oil (w/o) emulsions can be formed wherein the external phase is oleaginous in character. Preservatives/bacteriostats, such as the parabens, buffer systems, etc. can be incorporated into these bases as emulsified aqueous solutions together with the active ingredient. Diverse additives are conveniently used as the emulsifier, and these include, but are not limited to, cholesterol, lanolin (which contains cholesterol and cholesterol esters and other emulsifiers), lanolin derivatives, beeswax, fatty alcohols, wool wax alcohols, low HLB (hydrophobellipophobe balance) emulsifiers, and assorted ionic and nonionic surfactants, singularly or in combination.

C. Wound Dressings and Formulations

Articles containing the disclosed miRNA inhibitors for application to wounds are also provided. For example, transdermal patches, dressings, pads, wraps, matrices and bandages are provided that are capable of being adhered or otherwise associated with the skin of a subject and capable of delivering a therapeutically effective amount of one or more disclosed miRNA inhibitors.

The miRNA inhibitors can be impregnated in wound dressings known in the art of wound healing such as, but not necessarily restricted to, a cream, ointment, gel, solution, lotion, liniment, viscous emulsion, powder, paste, beads, a film dressing such as polyurethane film, a foam dressing such as a polyethane or polyurethane foam dressing, a hydrocolloid dressing, a hydrogel, alginate, gauze, paraffin gauze, hypertonic-saline-gauze, wet-dry-saline gauze, continuously-moist-saline gauze, expanding dressings, or Silver nanotech. The selection of dressing depends on the specific condition, grade, description, characteristics and bacterial profile.

D. Kits

The formulations may be provided as a kit or other container. The kit or container holds an effective amount of miRNA antagonist as defined herein. In some embodiments the composition is provided as part of a bandage. For example, the compositions can be applied to one side of a bandage or a transdermal patch, or the bandage or patch can be saturated with a liquid suspension of the composition.

Liquid compositions containing miRNA antagonists can be administered from absorbent materials, such as a bandage, patch or sponge, or as a spray or aerosol (applied to the affected area using a pump-type or aerosol sprayer). The use of a patch or bandage, into which the composition has been incorporated, is advantageous in that it the composition will be slowly and continuously released. Providing the composition in the form of a solution, which may initially be provided in a concentrated liquid form, or as a sterile dissolvable powder, for example, in a packet or syringe, requiring the addition of water, saline or other suitable diluents prior to use may be advantageous.

Solid compositions can be applied by any number of means, including the use of applicators or by patient self-administration. For example, creams, lotions, foams, pastes, ointments, or gels may be administered using an applicator, such as a squeeze-type or plunger-type applicator. Administering the composition as a cream having low surface tension is advantageous as it provides a uniform wetting action that assists in composition penetration into crypts and crevices of the wound. Such a creamy composition can also act as a moisturizer.

### II. Methods of Use

A. Promotion of Wound Healing

The disclosed miRNA antagonists can be used to promote wound healing in both chronic non-healing, and acute wounds. The wound being treated can be an ischemic wound. The wound being treated can be an non-ischemic wound.

The examples below demonstrate a general role for miRNAs in regulation of re-epithelialization of wounds. Therefore, it is expected that miRNA antagonists will be effective to promote wound healing in many epithelial tissues, including skin, mouth tissue, gingiva and corneal epithelium.

1. Chronic Wounds

Wound healing involves a complex interaction between epidermal and dermal cells, the extracellular matrix, controlled angiogenesis, and plasma derived proteins, all coordinated by an array of cytokines and growth factors. This dynamic process has been classically divided into several overlapping phases: inflammation, proliferation, migration and remodeling.

The combination of new tissue and contraction of surrounding tissues is essential for the healing of chronic skin ulcers (Clark, Dermatol. Clin., 11:657-666 (1993)). Fibroblasts are the key cells involved in the production of new extracellular matrix (in addition to producing collagen they produce tenascin, fibronectin, and proteoglycans such as hyaluronic acid). While this new matrix is synthesized, existing matrix in and around the wound region is degraded by several enzyme systems, including matrix metalloproteinases and plasminogen activators. The effect of metalloproteinases is regulated by tissue inhibitors, which are believed to be important in healing by preventing excessive matrix degradation (March, et al., Arch. Dermatol. Res., 287:107-114 (1994)). At an injury site, keratinocytes are also a part of the primary response to injury, releasing a signal and mobilizing other cell types (macrophages, platelets, endothelial cells and fibroblasts) to the site of injury. In addition, keratinocytes respond to cellular signals by undergoing two processes: migration and proliferation. Both of these processes are important for complete epithelialization and wound closure. During healing, some keratinocytes at the wound edge proliferate. Others undergo a marked transformation to enable them to phagocytose debris and migrate across the wound bed. Keratinocyte migration, coupled with wound contraction, results in re-epithelialization and wound closure. However, the epidermal morphology of chronic wounds differs from the morphology of normal epidermis and suggests that keratinocytes do not successfully complete activation or differentiation in chronic wounds (Stojadinovic, et al., Am. J. Pathol., 167:59-69 (2005); Morasso, et al., Biol. Cell, 97:173-183 (2005)). Instead, keratinocytes are caught in a “loop” of trying, but not succeeding, to accomplish either of the two processes. Keratinocytes at the non-healing edge of chronic wounds appear to be hyperproliferative but non-migratory, suggesting that lack of migration leads to inability to epithelialize and plays important role in pathogenesis of chronic ulcers.

The examples below demonstrate that miRNAs are overexpressed in epithelial cells at non-healing edges of chronic, non-healing wounds as compared to epithelial cells from adjacent healthy skin. The examples below also demonstrate that miRNAs overexpressed in chronic wounds target mRNAs that encode proteins that function in wound healing and inhibit acute wound healing in organ culture wound models. It is believed that overexpression of these miRNAs contributes to attenuation of growth factor signaling and general transcriptional attenuation that occurs in chronic non-healing wounds.

Methods for using the disclosed miRNA antagonists to inhibit or reduce the expression of miRNAs that are overexpressed in chronic non-healing wounds and to promote wound healing are provided. In one embodiment, the miRNA antagonists are administered to an individual in an effective amount to inhibit or reduce the expression of miRNAs that are overexpressed in chronic non-healing wounds.

In another embodiment, the miRNA antagonists are administered to an individual in an effective amount to promote wound healing. As used herein, the phrases “promote wound healing” or “promote wound closure” refer to increasing keratinocyte migration in a wound, reducing the amount of time required for a wound to close, increasing the extent to which a wound closes, or a combination thereof.

miRNA antagonists may be administered in any combination. For example, one or more miRNA antagonists that target the same miRNA that is overexpressed in chronic wounds may be co-administered. Alternatively, one or more miRNA antagonists that target different miRNAs that are overexpressed in chronic wounds may be co-administered.

2. Acute Wounds

The examples demonstrate that miRNAs are overexpressed in at non-healing edges of non-healing wounds as compared to epithelial cells from adjacent healthy skin. The examples also demonstrate that miRNAs that are induced in chronic wounds target mRNAs that encode proteins that function in wound healing generally and inhibit acute wound healing in organ culture wound models. Therefore, it is expected that miRNAs play important roles in both chronic non-healing wounds and in acute wounds and that miRNA antagonists can be used to promote healing in both of these types of wounds.

In one embodiment, the wound that is treated is a chronic non-healing wound. Representative chronic non-healing wounds that can be treated include, but are not limited to, diabetic ulcers, arterial ulcers, venous ulcers, pressure (decubitus) ulcers and burns.

In another embodiment, the wound that is treated using the disclosed miRNA antagonists is an acute wound, such as a wound caused by acute injury or surgery.

3. Infected Wounds

The examples demonstrate that miRNAs are overexpressed in infected wounds. For example, infection of porcine wounds with biofilm-forming Pseudomonas induced miR-21 and miR-27b expression compared to control wounds. Therefore, it is expected that miRNAs play important roles in wound infected with bacteria, such as biofilm bacteria, and that miRNA antagonists can be used to promote healing in these infected wounds.

Bacterial biofilms can impair cutaneous wound healing and reduce topical antibacterial efficiency in healing or treating infected skin wounds. Biofilm bacteria are less susceptible to our immune defense system, and consequently, a biofilm-associated infection can persist for a long period of time (i.e., progress from an acute to a chronic infection).

In one embodiment, the wound that is treated is infected with a biofilm bacteria such as Pseudomonas aeruginosa.

B. Treatment of Other Inflammatory Disorders

Inflammation is a critical component to wound healing. With the wounding of healthy tissue, a predictable progression of physiologic events unfolds. This progression can be divided into the phases of inflammation, proliferation, and maturation. The inflammatory phase simultaneously launches hemostatic mechanisms and pathways that create the clinically recognizable cardinal signs of inflammation: rubor (redness), calor (warmth), tumor (swelling), dolor (pain), and functio laesa (loss of function).

Chronic inflammation is a characteristic of chronic, non-healing wounds. It is known that molecular events that occur in chronic wounds are shared by other inflammatory conditions. For example, pro-inflammatory cytokines such as IL-1, TNFα are induced and persistent in chronic wound environment (Barrientos, et al., Wound Repair Regen., 16(5):585-601 (2008)). Therefore, it is expected that miRNAs that are upregulated in chronic wounds are also upregulated in other inflammatory conditions and contribute to the development and/or progression of these conditions.

In some embodiments, the miRNA antagonists are used to treat inflammatory conditions including, but are not limited to, arthritis, osteoarthritis, rheumatoid arthritis, asthma, vasculitis, inflammatory bowel disease, pelvic inflammatory disease, inflammatory skin disorders, multiple sclerosis, osteoporosis, tendonitis, allergic disorders, sepsis, atherosclerosis, and systematic lupus erythematosus.

C. Methods of Administration

The disclosed miRNA antagonists can be administered topically or subcutaneously at or adjacent to the site of a wound. In a preferred embodiment, the miRNA antagonists are administered topically. Topical administration may be in any suitable form, such as liquids, ointments, lotions, creams, gels, drops, sprays, patches or powders, as described above. The miRNA antagonists may also be incorporated into inserts, wound dressings, or other materials that come into contact with the wound.

The miRNA antagonists may be administered using any method that facilitates transdermal delivery. For example, the miRNA antagonists may be administered using compositions and methods that open channels within the stratum corneum, including, but not limited to laser assisted delivery (LAD), tape stripping, and cold plasma.

The miRNA antagonists may be administered using laser assisted delivery (LAD). Generally, in LAD, a pulsed laser removes micrometers (μm) of the stratum corneum per pulse. The laser can stop at the start of wet viable epidermis and not violate the skin's blood vessels, so there is no bleeding. The hole created in the stratum corneum can then facilitate delivery of drugs or collection of biochemical from the skin site. See U.S. Pat. No. 5,713,845 to Tankovich for example. Tankovich provides a method that involves administering an miRNA antagonists and a explosive absorber of light energy topically to the skin. The treated area of the skin can be illuminated with very short pulses of light which is preferentially absorbed by the absorber causing a very large number of tiny explosions. The tiny explosion forces portions of the drug to penetrate into the skin.

The miRNA antagonists may be administered using tape stripping. A tape stripping method typically involves applying an adhesive tape to the skin of a subject and removing the adhesive tape from the skin of the subject one or more times. In certain examples, the adhesive tape is applied to the skin and removed from the skin about one to ten times. Alternatively, about ten adhesive tapes can be applied to the skin and removed from the skin.

The miRNA antagonists may be administered using electroporation. For example, the miRNA antagonists are administered to the skin and a pulsed electric field applied to the skin to cause electrotransport of the miRNA antagonists into cells of the skin. See U.S. Pat. No. 6,520,950 to Hofmann et al. for an exemplary method of using electroporation to administer polynucleotide agents.

The miRNA antagonists may be for formulated into sustained release formulations such as polymeric delivery systems, mini-pumps, and hydrogels, as described above. These can be loaded with miRNA antagonists, injected or implanted into the ulcers, where the miRNA antagonists are released over a therapeutically effective time period.

For the treatment of inflammatory conditions not involving the skin or other exposed epithelial surface, the miRNA antagonists may be administered by a number of routes including, but not limited to, oral, inhalation (nasal or pulmonary), intravenous, intraperitoneal, intramuscular, sublingual, or rectal means. Injections can be e.g., intravenous, intradermal, subcutaneous, intramuscular, or intraperitoneal. In some embodiments, the injections can be given at multiple locations. The administration may be localized (i.e., to a particular region, physiological system, tissue, organ, or cell type) or systemic.

The miRNA antagonists may be administered in a single dose, in multiple doses, in a continuous or intermittent manner, depending, for example, upon the recipient's physiological condition. Administration of the compositions may be essentially continuous over an indeterminate period of time, for example, at regular intervals. Alternatively, the compositions can be administered continuously for a pre-selected period of time or in a series of spaced doses.

1. Effective Amounts

Certain factors may influence the dosage required to effectively treat a subject, including, but not limited to, the severity of the disease or disorder, previous treatments, the general health and/or age of the subject, and other diseases present. The effective dosage of the miRNA antagonist used for treatment may increase or decrease over the course of a particular treatment. Changes in dosage may result and become apparent from the results of diagnostic assays. For example, the subject can be monitored after administering an miRNA antagonist composition. Based on information from the monitoring, an additional amount of the miRNA antagonist composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀s found to be effective in in vitro and in vivo animal models.

Dosage levels on the order of about 1 μg/kg to 100 mg/kg of body weight per administration are useful in the treatment of a disease. One skilled in the art can also readily determine an appropriate dosage regimen for administering the disclosed to a given subject. For example, the miRNA antagonist composition can be administered to the subject once, e.g., as a single injection. Alternatively, the miRNA antagonist composition can be administered once or twice daily to a subject for a period of from about three to about twenty-eight days, or from about seven to about ten days.

Thus, the miRNA antagonist composition can be administered at a unit dose less than about 75 mg per kg of bodyweight, or less than about 70, 60, 50, 40, 30, 20, 10, 5, 2, 1, 0.5, 0.1, 0.05, 0.01, 0.005, 0.001, or 0.0005 mg per kg of bodyweight, and less than 200 nmol of miRNA antagonist per kg of bodyweight, or less than 1500, 750, 300, 150, 75, 15, 7.5, 1.5, 0.75, 0.15, 0.075, 0.015, 0.0075, 0.0015, 0.00075, 0.00015 nmol of miRNA antagonist per kg of bodyweight.

Where a dosage regimen comprises multiple administrations, it is understood that the effective amount of miRNA antagonist administered to the subject can include the total amount of miRNA antagonist administered over the entire dosage regimen. One skilled in the art will appreciate that the exact individual dosages may be adjusted somewhat depending on a variety of factors, including the specific miRNA antagonist being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. For instance, oral administration generally would be expected to require higher dosage levels than administration by intravenous injection. Variations in these dosage levels can be adjusted using standard empirical routines of optimization, which are well-known in the art. The precise therapeutically effective dosage levels and patterns are preferably determined by the attending physician in consideration of the above-identified factors.

In one embodiment, the unit dose is administered less frequently than once a day, e.g., less than every 2, 4, 8 or 30 days. In another embodiment, the unit dose is not administered on a regular schedule. For example, the unit dose may be administered a single time. Because oligonucleotide agent-mediated silencing can persist for several days after administering the miRNA antagonist composition, in many instances, it is possible to administer the composition with a frequency of less than once per day, or, for some instances, only once for the entire therapeutic regimen.

In some embodiments, a subject is administered an initial dose, and one or more maintenance doses of an miRNA antagonist composition. The maintenance dose or doses are generally lower than the initial dose, e.g., one-half less of the initial dose. A maintenance regimen can include treating the subject with a dose or doses ranging from 0.01 μg to 75 mg/kg of body weight per day, e.g., 70, 60, 50, 40, 30, 20, 10, 5, 2, 1, 0.5, 0.1, 0.05, 0.01, 0.005, 0.001, or 0.0005 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

Effective dosages can also be determined by extrapolation based on animal studies, for example, using a mouse model. The C57BL/KsJ db/db mouse is a particularly useful model since it has been shown to be a clinically relevant model of impaired wound healing. The animals exhibit several characteristics of adult onset diabetes, including obesity, insulin-resistant hyperglycemia and markedly delayed wound closure. C57BL/KsJ-db/db mice, homozygous for the diabetes spontaneous mutation, become identifiably obese around 3 to 4 weeks of age. Elevations of plasma insulin begin at 10 to 14 days and of blood sugar at 4 to 8 weeks. Homozygous mutant mice are polyphagic, polydipsic, and polyuric. The course of the disease is markedly influenced by genetic background. A number of features are observed on the C57BLIKsJ db/db background, including an uncontrolled rise in blood sugar, severe depletion of the insulin-producing beta-cells of the pancreatic islets, and death by 10 months of age. Exogenous insulin fails to control blood glucose levels and gluconeogenic enzyme activity increases. The diabetic mutation is a result of a point mutation in the leptin receptor gene, lepr. This point mutation promotes abnormal splicing creating a stop codon that shortens the intracellular domain of the receptor, so that its signaling capacity is curtailed. The ligand, Leptin, has been shown to be a key weight control hormone that takes a mutant form in the mouse obesity mutation, Lepob (JAX Mice database: http://jaxmice.jax.org/jaxmic-e-cgi/jaxmicedb.cgi).

C57BLJKsJ-db/dbmice exhibit characteristics similar to those of human adult onset diabetes (NIDDM Type 11) as a result of a single autosomal recessive mutation on chromosome 4. Only the homozygous animals develop diabetes. This strain also expresses lower levels of several growth factors and receptors, accounting, at least in part, for the reduced rate of healing (Werner, et al., J Invest Dermatol, 103:469-473 (1994)).

The streptozotocin diabetic mouse is another model for studying the pathology of diabetes. Mice are rendered diabetic by intraperitoneal injection of streptozotocin administered for five consecutive days. Streptozotocin-treated mice become hyperglycemic and also show impaired wound healing when compared to healthy animals (Matsuda, et al. J Exp Med, 187:297-306 (1998); Brown, et al., Am J Pathol, 151:715-724 (1997)). The streptozotocin-induced diabetic mouse has been widely studied and is known to those of skill in the art.

The diabetic mouse model (Geerlings, et al., FEMS Immunol Med Microbial., 3-4:259-265 (1999); Feige, et al., EXS., 77:359-373 (1996); Bessman, J Diabetes Complications, 4:258-262 (1992); Loots, et al., J Invest Dermatol., 5:850-857 (1998); Brown, et al., J Surg Research, 56:562-570 (1994); Greenhalgh, et al., Am J Pathol, 136:1235-1246 (1990); Tsuboi, et al., J Explorer Med, 172:245-251 (1990); Matuxzewska, et al., Pharm Res, 11:65-71 (1994); Darby, et al., Int J Biochem Cell Biol, 29:191-200 (1997); Livant, et al., J Clin Invest., 105:1537-1545 (2000); Yamamota, et al., Europ J Pharm, 302:53-60 (1996); Wetzler, et al., J Invest Dermatol., 115:245-253 (2000); Sun, et al., J Invest Dermatol, 108:313-318 (1997); Sun, et al., J Invest Dermatol., 106:232-237 (1996); Zykova, et al., Diabetes, 49:1461-1458 (2002); Beer, et al., J Invest Dermatol., 109: 132-138 (1997)) has been widely accepted in the study of therapeutic agents that may be effective in the treatment of chronic wounds, it has been successfully used in preclinical testing for growth factor therapies, and it offers a good model for patients with diabetic foot ulcers and other chronic, non-healing wounds.

D. Combination Therapies

Other therapeutic agents, for example, anti-microbial agents, pain relievers, anti-inflammatory agents, growth factors (e.g., PDGF), vitamins (e.g., vitamin B, C or E), aloe Vera or similar materials, may also be administered. Anti-miRNA molecules can also be applied in combination with other skin treatments such as an exfolliant or laser treatment.

### EXAMPLES

### Example 1

### Transcriptional Suppression in Venous Ulcers (VUs)

Materials and Methods:

Skin Specimens Used in Study

Institutional review board approval was obtained (approved protocol 01-0960(001) 03sux) and skin biopsies deriving from non-healing edges of chronic wounds were collected from discarded tissue after surgical debridement procedures on three consenting patients with venous reflux ulcers. Three normal skin specimens were obtained as discarded tissue from voluntary corrective surgery (approved protocol #25121). A small portion of skin biopsies were embedded in OCT compound (Tissue Tek) and frozen in liquid nitrogen at the same time as majority of the samples were stored in RNAlater (Ambion) for the subsequent RNA isolation. Before RNA was isolated from skin specimens H&E staining was performed to check on tissue morphology. All specimens showed hyperproliferative, hyper and para-keratotic epidermis typical for non-healing edges of chronic ulcers. To address mixed cell population the signal intensity levels in all chronic wound biopsies were compared, which correlate to the hybridization signal intensity of vimentin and perlecan for fibroblasts and stratifin and junctional plakoglobin for keratinocytes and found all four genes highly expressed at similar levels which means that both cell types are present in similar numbers.

Preparation and Hybridization of Probes

Specimen samples were homogenized and total RNA was isolated using RNeasy Mini Kit (Qiagen, Valencia, Calif.) according to the manufacturer's protocol. Approximately 5 μg of total RNA was reverse transcribed, amplified, and labeled as described (Stojadinovic, et al., J. Biol. Chem., 282:4021-4034 (2007)). Labeled cRNA was hybridized to HG-U133A-set Gene Chip probe arrays that contain probe sets representing approximately more than 22000 genes (Affymetrix, Santa Clara, Calif.). Arrays were washed, stained with avidin-biotin streptavidin-phycoerythin labeled antibody using Affymetrix fluidics station and then scanned using the Agilent GeneArray Scanner system (Hewlett-Packard, Palo Alto, Calif.) as described by Affimetrix.

Gene Array Data Analysis

Microarray Suite 5.0 (Affymetrix) was used for data extraction and for further analysis, data mining tool 3.0 (Affymetrix, Santa Clara, Calif.) and GeneSpring™ software 7.3.1 (Silicon Genetics, Redwood City, Calif.) was used for normalization and filtering on Volcano plot for fold change and p-value calculations. Samples were normalized per chip and per gene. Statistical comparisons of expression level, between each condition were performed using ANOVA test. Only genes with a p-value less than 0.05 were considered to be statistically significant. Differential expressions of transcripts were determined by calculating the fold change. Genes were considered regulated if the expression levels differed more than 2-fold relative to normal skin. Clustering was performed based on individual genes expression profiles. An extensive gene annotation table was developed, describing the molecular function and biological category of the genes present on the chip based on Gene Ontology Consortium (cgap.nci.nih.gov/Genes/GOBrowser). The genes were annotated in Table 1.

Results:

To establish a pathogenic transcriptional profile, RNA was prepared from biopsies from non-healing edges of patients with venous ulcers and from healthy skin. Expression levels of mRNAs were compared between the samples using a microarray approach. A total of 1,411 differentially regulated genes were found using this analysis, 49% of which were suppressed in ulcers. In addition, 144 of the suppressed genes constitute genes that actively participate in transcription. For example, transcription factors such as GADD45B, JUN, JUNB, SOX9, etc. were found to be suppressed. Furthermore, regulatory molecules that transmit signal such as EGFR, MET, DTR, FGFR1 along with growth factors/cytokines were down-regulated, overall suggesting global transcriptional suppression in chronic wounds. The analysis did not indicate that any of the chromatin modulators (such as HATs, CARMs or HDACs) were differentially regulated between the samples, suggesting that chromatin modifications may not be participating in this transcriptional suppression.

It was postulated that miRNAs may participate in suppression of gene expression in chronic, non-healing wounds. Specifically, it was postulated that miRNAs that target growth factor/cytokines that participate in wound healing may be induced and overexpressed in chronic ulcers. To begin to explore the regulation of miRNAs in non-healing wounds, a computational approach was adopted to predict miRNAs that are upregulated in chronic, non-healing wounds.

A statistical analysis was then performed to screen for human miRNAs whose predicted targets were expressed at significantly lower levels in wound biopsies as compared to normal skin. This reduced mRNA expression signature for miRNA targets genes served as a statistical signal that the miRNAs may be aberrantly overexpressed in chronic wounds. As the goal is to infer the activity of miRNAs rather than to predict miRNA targets per se, an approach was taken of defining the set of putative targets of a miRNA to be all genes containing at least one 7-mer seed match (complementarity at miRNA positions 2-8 or positions 2-7 with nucleotide ‘A’ across from position 1 in the 3′ UTR (Grimson, et al., Mol. Cell, 27(1):91-105 (2007)). The results were also filtered for whether the seed class was conserved across vertebrates, since conserved miRNAs may play a more important functional role. Approximately 4950 genes expressed in chronic wounds were considered. Their log expression changes in wound biopsies versus normal skin were computed, and an assessment was performed as to whether each miRNA's target set was expressed at significantly lower levels, as a distribution, compared to all genes by computing a one-sided Kolmogorov-Smirnov test statistic. 152 human miRNA seed families (miRNA with identical seeds) were screened in this way a cut-off of p<0.0001 was used as a significance threshold.

In addition to looking for the target gene expression signature described above, a set of functional filters for relevance to wound repair was then applied. First, for a collection of wound repair-related functional annotations (e.g. receptor growth factor, cell proliferation), it was assessed whether (i) the functional class was over-represented in the miRNA target list and (ii) the median expression change of genes in the functional class was significantly lowered in chronic wounds. Second, it was checked whether miRNA profiling evidence from other studies supported the expression of the miRNA in epithelial cells (cell line MFC10A) or in psoriasis (Sonkoly, et al., PLoS ONE, 2(7):e610 (2007)). Finally, it was considered whether the seed class was conserved across vertebrates, since conserved miRNAs may play a more important functional role. The complete set of miRNAs predicted to be upregulated in chronic, non-healing wounds is presented in Table 1 below. miRNAs that are listed in Table 1 in bold font were also confirmed to be upregulated using quantitative PCR.

TABLE 1
miRNAs predicted to be upregulated in chronic, non-healing wounds.

| | | | | | Highly | MiRBase |
|---|---|---|---|---|---|---|
| miRNA | KS p-value | # genes | Targets growth factors? | Source | Conserved? | Accession No. |
| miR-17-5p | 2.65E−11 | 287 | Receptor growth factor | Sonkoly et al | YES | |
| miR-20a | | | | | | MI0000076 |
| miR-20b | | | | | | MI0001519 |
| miR-93.mr | | | | | | |
| miR-106a | | | | | | MI0000113 |
| miR-106b | | | | | | MI0000734 |
| miR-519.d | | | | | | |
| miR-181a-1 | 3.70E−11 | 253 | | MCF10A cell line | YES | MI0000289 |
| miR-181a-2 | | | | | | MI0000269 |
| miR-181b-1 | | | | | | MI0000270 |
| miR-181b-2 | | | | | | MI0000683 |
| miR-181c | | | | | | MI0000271 |
| miR-181d | | | | | | MI0003139 |
| miR-300 | 6.80E−10 | 211 | Receptor growth factor | Control | | MI0005525 |
| miR-381 | | | | | | MI0000789 |
| miR-9-1 | 2.00E−08 | 316 | | | YES | MI0000466 |
| miR-9-2 | | | | | | MI0000467 |
| miR-9-3 | | | | | | MI0000468 |
| miR-503 | 4.74E−08 | 89 | | | YES | MI0003188 |
| miR-543 | 6.37E−08 | 132 | | | | MI0005565 |
| miR-135a-1 | 2.07E−07 | 135 | Receptor growth factor | | YES | MI0000452 |
| miR-135a-2 | | | | | | MI0000453 |
| miR-135b | | | | | | MI0000810 |
| miR-106a | 2.99E−07 | 169 | Receptor growth factor | Sonkoly et al | YES | MI0000113 |
| miR-106b | | | | | | MI0000734 |
| miR-302a | | | | | | MI0000738 |
| miR-302b | | | | | | MI0000772 |
| miR-302c | | | | | | MI0000773 |
| miR-302d | | | | | | MI0000774 |
| miR-302e | | | | | | MI0006417 |
| miR-302f | | | | | | MI0006418 |
| miR-21 | 3.47E−07 | 64 | Receptor growth factor | Sonkoly et al, MCF10A cell line | YES | MI0000077 |
| miR-590-5p | | | | | | |
| miR-19a | 9.70E−07 | 279 | Receptor growth factor | | YES | MI0000073 |
| miR-19b-1 | | | | | | MI0000074 |
| miR-19b-2 | | | | | | MI0000075 |
| miR-101-1 | 1.04E−06 | 190 | | | YES | MI0000103 |
| miR-101-2 | | | | | | MI0000739 |
| miR-377 | 1.15E−06 | 102 | | | | MI0000785 |
| miR-218-1 | 1.31E−06 | 166 | Receptor growth factor | | YES | MI0000294 |
| miR-218-2 | | | | | | MI0000295 |
| miR-410 | 3.33E−06 | 135 | Receptor growth factor | | | MI0002465 |
| miR-15a | 5.83E−06 | 307 | Receptor growth factor | MCF10A cell line | YES | MI0000069 |
| miR-15b | | | | | | MI0000438 |
| miR-16-1 | | | | | | MI0000070 |
| miR-16-2 | | | | | | MI0000115 |
| miR-195 | | | | | | MI0000489 |
| miR-424 | | | | | | MI0001446 |
| miR-497 | | | | | | MI0003138 |
| miR-103-1 | 7.44E−06 | 142 | Receptor growth factor | | YES | MI0000109 |
| miR-103-2 | | | | | | MI0000108 |
| miR-103-1-as | | | | | | MI0007261 |
| miR-103-2-as | | | | | | MI0007262 |
| miR-107 | | | | | | MI0000114 |
| miR-96 | 7.79E−06 | 257 | | | YES | MI0000098 |
| miR-1271 | | | | | | MI0003814 |
| miR-144 | 7.83E−06 | 201 | | | YES | MI0000460 |
| miR-25 | 8.75E−06 | 210 | | | YES | MI0000082 |
| miR-32 | | | | | | MI0000090 |
| miR-92a-1 | | | | | | MI0000093 |
| miR-92a-2 | | | | | | MI0000094 |
| miR-92b | | | | | | MI0003560 |
| miR-363 | | | | | | MI0000764 |
| miR-367 | | | | | | MI0000775 |
| miR-137 | 1.35E−05 | 235 | | | YES | MI0000454 |
| miR-145 | 1.38E−05 | 177 | Receptor growth factor | | YES | MI0000461 |
| miR-130a | 1.51E−05 | 207 | Receptor growth factor | MCF10A cell line | YES | MI0000448 |
| miR-130b | | | | | | M10000748 |
| miR-301a | | | | | | MI0000745 |
| miR-301b | | | | | | MI0005568 |
| miR-374a | 1.69E−05 | 138 | | | | MI0000782 |
| miR-374b | | | | | | MI0005566 |
| miR-221 | 2.27E−05 | 93 | Receptor growth factor | | YES | MI0000298 |
| miR-222 | | | | | | MI0000299 |
| miR-200b | 2.28E−05 | 249 | | MCF10A cell line | YES | MI0000342 |
| miR-200c | | | | | | MI0000650 |
| miR-429 | | | | | | MI0001641 |
| miR-153-1 | 2.53E−05 | 155 | Receptor growth factor | | YES | MI0000463 |
| miR-153-2 | | | | | | MI0000464 |
| miR-219-1 | 3.06E−05 | 65 | | | YES | MI0000296 |
| miR-219-2 | | | | | | MI0000740 |
| miR-219-5p | | | | | | |
| miR-27a | 5.26E−05 | 255 | Receptor growth factor | MCF10A cell line | YES | MI0000085 |
| miR-27b | | | | | | MI0000440 |
| miR-340 | 6.15E−05 | 279 | | | | MI0000802 |
| miR-340-5p | | | | | | |
| miR-128-1 | 6.76E−05 | 216 | Receptor growth factor | | YES | MI0000447 |
| miR-128-2 | | | | | | MI0000727 |
| miR-23a | 9.81E−05 | 233 | | MCF10A cell line | YES | MI0000079 |
| miR-23b | | | | | | MI0000439 |
| miR-448 | 0.00011294 | 127 | Receptor growth factor | | | MI0001637 |
| miR-495 | 0.00012976 | 185 | | | | MI0003135 |
| miR-1192 | | | | | | |
| mIR-138-1 | 0.00014676 | 120 | | | YES | MI0000476 |
| miR-138-2 | | | | | | MI0000455 |
| miR-203 | 0.0001508 | 177 | | Sonkoly et al | YES | MI0000283 |
| miR-141 | 0.00015314 | 162 | Receptor growth factor | Sonkoly et al, MCF10A cell line | YES | MI0000457 |
| miR-200a | | | | | | MI0000737 |
| miR-494 | 0.00020123 | 100 | | | | MI0003134 |
| miR-29a | 0.0002124 | 266 | Receptor growth factor | MCF10A cell line | YES | MI0000087 |
| miR-29b-1 | | | | | | MI0000105 |
| miR-29b-2 | | | | | | MI0000107 |
| miR-29c | | | | | | MI0000735 |
| miR-148a | 0.00023714 | 166 | | | YES | MI0000253 |
| miR-148b | | | | | | MI0000811 |
| miR-152 | | | | | | MI0000462 |
| miR-24-1 | 0.00026661 | 132 | Receptor growth factor | MCF10A cell line | YES | MI0000080 |
| miR-24-2 | | | | | | MI0000081 |
| miR-758 | 0.00038025 | 49 | | | | MI0003757 |
| miR-326 | 0.00041845 | 80 | | | | MI0000808 |
| miR-330 | | | | | | MI0000803 |
| miR-330-5p | | | | | | |
| miR-224 | 0.00047851 | 81 | | | | MI0000301 |
| miR-132 | 0.00056051 | 78 | | | YES | MI0000449 |
| miR-212 | | | | | | MI0000288 |
| miR-18a | 0.00066862 | 68 | | | YES | MI0000072 |
| miR-18b | | | | | | MI0001518 |
| miR-124-1 | 0.00069127 | 422 | | | YES | MI0000443 |
| miR-124-2 | | | | | | MI0000444 |
| miR-124-3 | | | | | | MI0000445 |
| miR-506 | | | | | | MI0003193 |
| miR-184 | 0.00072078 | 13 | | | YES | MI0000481 |
| miR-183 | 0.00072083 | 99 | Receptor growth factor | | YES | MI0000273 |
| miR-99a | 0.00075938 | 13 | Receptor growth factor | | YES | MI0000101 |
| miR-99b | | | | | | MI0000746 |
| miR-100 | | | | | | MI0000102 |
| miR-339-5p | 0.00084515 | 32 | | | | |
| miR-186 | 0.00084649 | 152 | Receptor growth factor | | | MI0000483 |
| miR-592 | 0.00095135 | 43 | | | | MI0003604 |
| miR-599 | | | | | | MI0003611 |
| miR-455 | 0.0009824 | 42 | Receptor growth factor | | YES | MI0003513 |
| miR-455-5p | | | | | | |
| miR-129-1 | 0.00101593 | 106 | | | YES | MI0000252 |
| miR-129-2 | | | | | | MI0000473 |
| miR-129-5p | | | | | | |
| miR-139-5p | 0.00105856 | 79 | Receptor growth factor | | YES | |
| miR-346 | 0.00107064 | 23 | | | | MI0000826 |
| miR-370 | 0.00137647 | 68 | | | | MI0000778 |
| miR-204 | 0.00155298 | 129 | Receptor growth factor | | YES | MI0000284 |
| miR-211 | | | | | | MI0000287 |
| miR-205 | 0.00160039 | 98 | | MCF10A cell line | YES | MI0000285 |
| miR-376c | 0.00208934 | 45 | Receptor growth factor | | | MI0000776 |
| miR-1-1 | 0.00211794 | 224 | Receptor growth factor | | YES | MI0000651 |
| miR-1-2 | | | | | | MI0000437 |
| miR-206 | | | | | | MI0000490 |
| miR-192 | 0.00271769 | 33 | | | YES | MI0000234 |
| miR-215 | | | | | | MI0000291 |
| miR-590 | 0.00273727 | 206 | | | | MI0003602 |
| miR-590-3p | | | | | | |
| miR-133a-1 | 0.00334115 | 167 | Receptor growth factor | | YES | MI0000450 |
| miR-133a-2 | | | | | | MI0000451 |
| miR-133b | | | | | | MI0000822 |
| miR-196a-1 | 0.00347214 | 50 | Receptor growth factor | | YES | MI0000238 |
| miR-196a-2 | | | | | | MI0000279 |
| miR-196b | | | | | | MI0001150 |
| miR-544 | 0.00395843 | 97 | Receptor growth factor | | | MI00003515 |
| miR-216a | 0.00482721 | 51 | Receptor growth factor | | YES | MI0000292 |
| miR-216b | | | | | | MI0005569 |
| miR-30a | 0.00505643 | 297 | | Sonkoly et al, MCF10A cell line | YES | MI0000088 |
| miR-30a-5p | | | | | | |
| miR-30b | | | | | | MI0000441 |
| miR-30b-5p | | | | | | |
| miR-30c-1 | | | | | | MI0000736 |
| miR-30c-2 | | | | | | MI0000254 |
| miR-30d | | | | | | MI0000255 |
| miR-30e | | | | | | MI0000749 |
| miR-384-5p | | | | | | |
| miR-504 | 0.00507711 | 47 | | | | MI0003189 |
| miR-223 | 0.00748116 | 63 | | | YES | MI0000300 |
| miR-217 | 0.00792727 | 81 | Receptor growth factor | | YES | MI0000293 |
| miR-876-5p | 0.00970108 | 38 | | | | |
| miR-28 | 0.0.0102727 | 37 | | | | MI0000086 |
| miR-28-5p | | | | | | |
| miR-708 | | | | | | MI0005543 |
| miR-22 | 0.0116508 | 93 | Receptor growth factor | MCF10A cell line | YES | MI0000078 |
| miR-411 | 0.0119299 | 20 | | | | MI0003675 |
| miR-31 | 0.0128238 | 70 | | Sonkoly et al | YES | MI0000089 |
| miR-365-1 | 0.0129088 | 54 | | | YES | MI0000767 |
| miR-365-2 | | | | | | MI0000769 |
| miR-193a | 0.0140258 | 41 | | | YES | MI0000487 |
| miR-193b | | | | | | MI0003137 |
| miR-214 | 0.0140774 | 129 | | | YES | MI0000290 |
| miR-761 | | | | | | |
| miR-873 | 0.01472 | 73 | | | | MI0005564 |
| miR-7-1 | 0.0150244 | 101 | | | YES | MI0000263 |
| miR-7-2 | | | | | | MI0000264 |
| miR-7-3 | | | | | | MI0000265 |
| miR-208a | 0.0153328 | 30 | | | YES | MI0000251 |
| miR-208b | | | | | | MI0005570 |
| miR-338 | 0.0157934 | 62 | | | YES | MI0000814 |
| miR-338-3p | | | | | | |
| miR-376a-1 | 0.0174268 | 44 | Receptor growth factor | | | MI0000784 |
| miR-376a-2 | | | | | | MI0003529 |
| miR-376b | | | | | | MI0002466 |
| miR-376b-3p | | | | | | |
| miR-376c | | | | | | MI0000776 |
| miR-155 | 0.0199024 | 81 | | | YES | MI0000681 |
| miR-182 | 0.0202365 | 266 | | | YES | MI0000272 |
| miR-875-5p | 0.0205124 | 19 | Receptor growth factor | | | |
| miR-26a-1 | 0.0211057 | 196 | | MCF10A cell line | YES | MI0000083 |
| miR-26a-2 | | | | | | MI0000750 |
| miR-26b | | | | | | MI0000084 |
| miR-1297 | | | | | | MI0006358 |
| miR-142-3p | 0.0215277 | 84 | | Sonkoly et al | YES | |
| miR-33a | 0.0220102 | 79 | Receptor growth factor | | YES | MI0000091 |
| miR-33b | | | | | | MI0003646 |
| miR-329-1 | 0.0220861 | 57 | | | | MI0001725 |
| miR-329-2 | | | | | | MI0001726 |
| miR-362-3p | | | | | | |
| miR-491 | 0.0233019 | 26 | Receptor growth factor | | | MI0003126 |
| miR-491-5p | | | | | | |
| miR-421 | 0.0244541 | 559 | | | | MI0003685 |
| miR-34a | 0.0261954 | 128 | Receptor growth factor | | YES | MI0000268 |
| miR-34b | | | | | | MI0000742 |
| miR-34b-5p | | | | | | |
| miR-34c | | | | | | MI0000743 |
| miR-34c-5p | | | | | | |
| miR-449a | | | | | | MI0001648 |
| miR-449b | | | | | | MI0003673 |
| miR-449c | | | | | | |
| miR-699 | | | | | | |
| miR-125a | 0.0265386 | 175 | Receptor growth factor | | YES | MI0000469 |
| miR-125b-1 | | | | | | MI0000446 |
| miR-125b-2 | | | | | | MI0000470 |
| miR-351 | | | | | | |
| miR-505.hm | 0.0266806 | 52 | | | | |
| miR-149 | 0.0286405 | 81 | | | | MI0000478 |
| miR-335 | 0.0352866 | 41 | | | | MI0000816 |
| miR-335-5p | | | | | | |
| miR-342 | 0.0358495 | 49 | | | | MI0000805 |
| miR-342-3p | | | | | | |
| miR-490 | 0.0391851 | 27 | | | YES | MI0003125 |
| miR-490-3p | | | | | | |
| miR-136 | 0.0407096 | 38 | | | | MI0000475 |
| let-7a-1 | 0.0.0409881 | 192 | Receptor growth factor | MCF10A cell line | YES | MI0000060 |
| let-7a-2 | | | | | | MI0000061 |
| let-7a-3 | | | | | | MI0000062 |
| let-7b | | | | | | MI0000063 |
| let-7c | | | | | | MI0000064 |
| let-7d | | | | | | MI0000065 |
| let-7e | | | | | | MI0000066 |
| let-7f-1 | | | | | | MI0000067 |
| let-7f-2 | | | | | | MI0000068 |
| let-7g | | | | | | MI0000433 |
| let-7i | | | | | | MI0000434 |
| let-98 | | | | | | |
| miR-378 | 0.0411793 | 33 | Receptor growth factor | | | MI0000786 |
| miR-422a | | | | | | MI0001444 |
| miR-361 | 0.0420349 | 32 | Receptor growth factor | | | MI0000760 |
| miR-361-5p | | | | | | |
| miR-320a | 0.047513 | 179 | Receptor growth factor | | | MI0000542 |
| miR-320b-1 | | | | | | MI0003776 |
| miR-320b-2 | | | | | | MI0003839 |
| miR-320c-1 | | | | | | MI0003778 |
| miR-320c-2 | | | | | | MI0008191 |
| miR-320d-1 | | | | | | MI0008190 |
| miR-320d-2 | | | | | | MI0008192 |
| miR-486 | 0.0485851 | 31 | | | | MI0002470 |
| miR-486-5p | | | | | | |
| miR-140 | 0.0492961 | 90 | | | YES | MI0000456 |
| miR-140-5p | | | | | | |
| miR-876-3p | | | | | | |
| miR-375 | 0.0552852 | 29 | Receptor growth factor | | YES | MI0000783 |
| miR-143 | 0.0603871 | 86 | | Control | YES | MI0000459 |
| miR-488 | 0.0663319 | 56 | | | | MI0003123 |
| miR-199a-1 | 0.0675119 | 123 | | | YES | MI0000242 |
| miR-199a-2 | | | | | | MI0000281 |
| miR-199b | | | | | | MI0000282 |
| miR-199-5p | | | | | | |
| miR-216a | 0.0758677 | 42 | | | YES | MI0000292 |
| miR-216b | | | | | | MI0005569 |
| miR-485 | 0.0830873 | 52 | | | | MI0002469 |
| miR-485-5p | | | | | | |
| miR-190 | 0.0841059 | 26 | | | YES | MI0000486 |
| miR-328 | 0.0856876 | 28 | | | | MI0000804 |
| miR-299 | 0.0948232 | 16 | | | | MI0000744 |
| miR-299-3p | | | | | | |
| miR-197 | 0.0968162 | 45 | | | | MI0000239 |
| miR-383 | 0.104534 | 27 | | | YES | MI0000791 |
| miR-382 | 0.112977 | 36 | | | | MI0000790 |
| miR-194-1 | 0.122032 | 85 | Receptor growth factor | | YES | MI0000488 |
| miR-194-2 | | | | | | MI0000732 |
| miR-10a | 0.123758 | 62 | | | YES | MI0000266 |
| miR-10b | | | | | | MI0000267 |
| miR-202 | 0.124731 | 144 | Receptor growth factor | | | MI0003130 |
| miR-202-3p | | | | | | |
| miR-384 | 0.129141 | 52 | | | | MI0001145 |
| miR-384-3p | | | | | | |
| miR-542 | 0.147684 | 57 | | | | MI0003686 |
| miR-542-3p | | | | | | |
| miR-499 | 0.152405 | 60 | | | YES | MI0003183 |
| miR-499-5p | | | | | | |
| miR-154 | 0.159075 | 31 | | | | MI0000480 |
| miR-296 | 0.16196 | 12 | | | | MI0000747 |
| miR-296-3p | | | | | | |
| miR-431 | 0.163445 | 25 | Receptor growth factor | | | MI0001721 |
| miR-539 | 0.190377 | 93 | | | | MI0003514 |
| miR-425 | 0.202205 | 40 | | | YES | MI0001448 |
| miR-489 | | | | | | MI0003124 |
| miR-290-5p | 0.234824 | 69 | | | | |
| miR-292-5p | | | | | | |
| miR-371-5p | | | | | | |
| miR-551a | 0.267157 | 3 | Receptor growth factor | | YES | MI0003556 |
| miR-551b | | | | | | MI0003575 |
| miR-450a-1 | 0.283382 | 1 | | | | MI0001652 |
| miR-450a-2 | | | | | | MI0003187 |
| miR-450b | | | | | | MI0005531 |
| miR-450a-5p | | | | | | |
| miR-379 | 0.299503 | 20 | | | | MI0000787 |
| miR-125a-3p | 0.322583 | 30 | Receptor growth factor | | | |
| miR-134 | 0.337787 | 29 | Receptor growth factor | | | MI0000474 |
| miR-191 | 0.414191 | 12 | | | YES | MI0000465 |
| miR-496 | 0.426379 | 22 | | | | MI0003136 |
| miR-433 | 0.428187 | 50 | Receptor growth factor | | | MI0001723 |
| miR-210 | 0.450674 | 5 | | | YES | MI0000286 |
| miR-150 | 0.49431 | 40 | | | YES | MI0000479 |
| miR-487a | 0.509368 | 2 | Receptor growth factor | | | MI0002471 |
| miR-487b | | | | | | MI0003530 |
| miR-185 | 0.546133 | 39 | | | | MI0000482 |
| miR-882 | | | | | | |
| miR-324-5p | 0.548534 | 25 | | | | |
| miR-146a | 0.584773 | 39 | | Sonkoly et al | YES | MI0000477 |
| miR-146b | | | | | | MI0003129 |
| miR-653 | 0.625017 | 30 | Receptor growth factor | | | MI0003674 |
| miR-451 | 0.713806 | 5 | | | YES | MI0001729 |
| miR-122 | 0.733976 | 40 | | | YES | MI0000442 |
| miR-126 | 0.871356 | 8 | Receptor growth factor | | YES | MI0000471 |
| miR-126-3p | | | | | | |
| miR-874 | 0.944414 | 33 | | | | MI0005532 |

### Example 2

### Specific miRNAs are Induced in Venous Ulcers

Materials and Methods:

RNA Isolation and Quantitative Real-Time PCR

Biopsies obtained after surgical debridement were collected immediately following surgery from 7 patients with venous ulcers (VUs). All biopsies were verified for established histological criteria for non-healing edges and nuclear presence of pathogenic marker β-catenin (Stojadinovic, et al., Am. J. Pathol., 167(1):59-69 (2005)). Total RNA with miRNA fraction was isolated using the miRVana RNA isolation Kit (Ambion). Detection and quantification of specific miRNAs was performed using TaqMan® MicroRNA Assays (Applied Biosystems). Target miRNA expression was normalized among different samples based on the values of U48 RNA expression. 100 ng of template RNA was reverse transcribed using the TaqMan® MicroRNA Reverse Transcription Kit and miRNA-specific stem-loop primers (Applied Biosystems). 1.33 μl of the reverse transcription product was then introduced into 20 μl PCR reactions which were incubated in 96-well plates to a thermocycler (MJ Research PTC-200) (BioRad) at 95° C. for 10 minutes, followed by 40 cycles of 95° C. for 15 seconds and 60° C. for 1 minute.

Cell Culture

Human epidermal keratinocytes were grown in defined serum-free keratinocyte medium supplemented with epidermal growth factor and bovine pituitary extract (keratinocyte-SFM, GIBCO). Cells were expanded through two 1:4 passages before transfection and transfected at approximately 70% confluence. Twenty-four hours before transfection, cells were incubated in basal keratinocyte medium (GIBCO) that was custom made without phenol red, hydrocortisone or thyroid hormone.

DNA Cloning and Transfection

A 227-bp DNA fragment flanking the pre-miR-21 hairpin was cloned from human genomic DNA using the following primers:

(SEQ ID NO: 5)
5′ primer: 5′-CGGGATCCTTATCAAATCCTGCCTGACTG-3′
(SEQ ID NO: 6)
3′ primer: 5′-CCCAAGCTTGACCAGAGTTTCTGATTATAACA-3′.

A 328-bp DNA fragment flanking the pre-miR-130a hairpin was cloned from human genomic DNA using the following primers:

(SEQ ID NO: 7)
5′ primer: 5′-CGGGATCCGCTGTATTGAAGCAAAGAAGG-3′
(SEQ ID NO: 8)
3′ primer: 5′-CCCAAGCTTGGGTAGCTGACTGGTGCC-3′.

The resulting pre-miR-21 and pre-miR-130a fragments were restricted and inserted into the BamHI and HindIII sites of pSilencer 4.1-CMV puro vector (Ambion, Applied Biosystems). A leptin receptor (LepR) 3′ UTR fragment containing miR-130a and miR-21 putative target sites was amplified from human genomic DNA. The primers used to amplify the LepR 3′ UTR fragment were the following:

(SEQ ID NO: 9)
5′ primer: 5′-gctctagaAGTCTAATCATGATCACTACAGATG-3′
(SEQ ID NO: 10)
3′ primer: 5′-gctctagaGAAAAATCCTGCCAAACAACTAC-3′.

The same fragment from the LepR 3′ UTR was also amplified in the antisense orientation for use as a control. The primers used to amplify the antisense LepR 3′ UTR fragment were the following:

5′ primer:
(SEQ ID NO: 11)
5′-gctctagaTTTCACTGAAGAAACCTTCAGATTTGTG-3′
3′ primer:
(SEQ ID NO: 12)
5′-GCTCTAGAggatttaaatctgaactctctgattcc-3′.

These fragments were inserted into the XbaI site in the 3′UTR of pGL3-control plasmid (Promega) and sequenced.

Statistical Analysis

Unpaired student t-test was performed and the P value was calculated for each experiment. For all experiments with error bars, standard deviation was calculated to indicate the variation within each experiment.

In Situ Hybridization

In situ transcriptional levels of miR-21 and miR-130a were determined on frozen sections (10 μm) of skin biopsy specimens from seven patients with chronic venous ulcers and four healthy individuals according to the manufacturer's instructions (Exiqon). Sections were hybridized overnight with digoxygenin-labeled miRCURY LNA probes (Exiqon) and incubated with anti-digoxygenin antibody conjugated with alkaline phosphatase for 1 hour. Sections were visualized by using BM purple substrate together with 2 mM levamisole using the protocol recommended by the manufacturer (Exiqon). The stained sections were viewed with a Zeiss microscope.

Results:

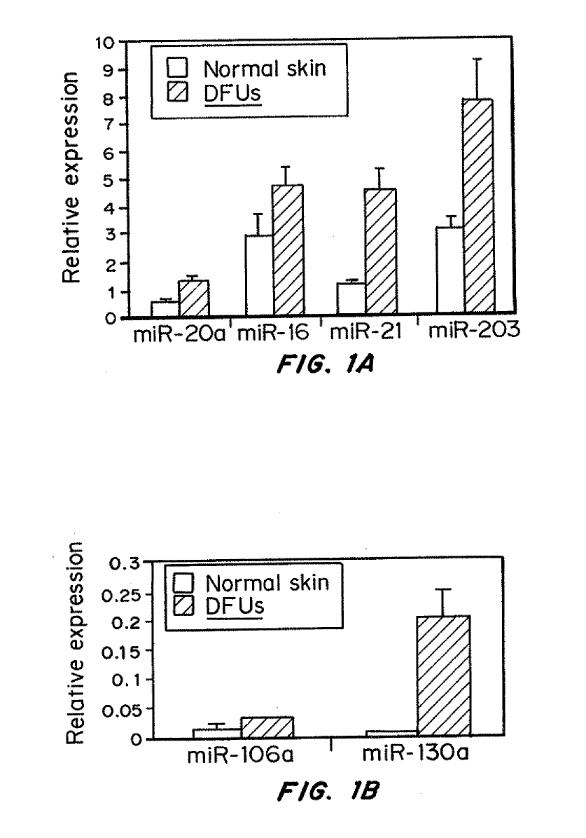

To analyze the results obtained by computational predictions in Example 1, quantitative real-time PCR (qRT-PCR) analysis was performed for selected miRNAs shown to be expressed in skin (Yi, et al., Nat. Genet., 38(3):356-62 (2006)). RNA samples were obtained from full thickness skin biopsies obtained after surgical debridement of VUs (n=7) and healthy skin (n=4). All biopsies were verified for established histological criteria for non-healing edges and nuclear presence of pathogenic marker β-catenin prior to RNA isolation (Stojadinovic, et al., Am. J. Pathol., 167(1):59-69 (2005)). Primers designed to specifically amplify the mature, active form of 12 miRNAs suggested to be the most unregulated in VUs by computational predictions were used (miR-381, miR-27a, miR-27b, miR-146a, miR-143, miR-17, miR-20a, miR-16, miR-21, miR-106a, miR-19a, miR-19b). Target miRNA expression was normalized among different samples based on the values of U48 RNA expression. qRT-PCR results showed significantly increased levels of hsa-miR-16, hsa-miR-21, hsa-miR-20a, hsa-miR-106a, has-miR-203 and hsa-miR-130a in chronic wounds when compared with healthy skin (FIGS. 1A and 1B). In particular, miR-130a and miR-21 were 58-fold and 5-fold induced, respectively. The striking overexpression of miR-130a and miR-21 suggests that they may play specific roles in the pathogenesis of chronic venous ulcers.

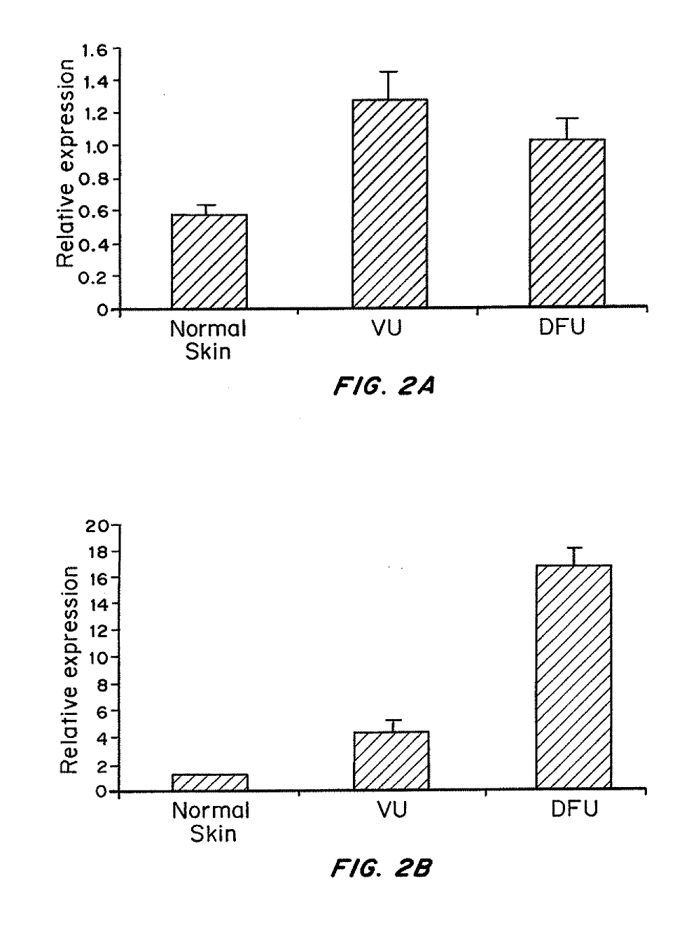

The previous studies were performed on samples from VUs. To test whether induction of miRNA plays a role in more general mechanisms of chronic ulcer pathology, this analysis was repeated on tissue specimens from DFU patients and qPCR analysis was used to confirm that miRNAs are indeed induced in DFUs (FIGS. 2A and 2B). The data confirmed that the first two miRNAs tested that were found to be overexpressed in VUs (miR-20a and miR-21) were also overexpressed in DFUs. These data suggest that induction of miRNA may contribute to overall inhibition of wound healing.

miR-21 was previously found to be upregulated both in psoriasis and atopic eczema (Sonkoly, et al., PLoS ONE, 2(7):e610 (2007)) and linked to invasion and metastasis of tumors, squamos cell carcinoma, and T-cell differentiation (Zhu, et al., Cell Res., 18(3):350-9 (2008); Wu, et al., PLoS ONE, 2(10):e1020 (2007) and angiogenesis (Chen and Gorski, Blood, 111(3):1217-26 (2008))). The role of miR-130a in skin and epithelial tissues has not been described before.

### Example 3

### miR21 and miR-130a Inhibit Acute Wound Healing

Materials and Methods:

Human Skin Organ Culture and Treatment with Mimics

Four healthy skin specimens were obtained as discarded tissue from patients undergoing elective plastic surgery and used for acute wounds as described by Tomic-Canic, et al., Wound Repair Regen., 15(1):71-9 (2007). Adipose tissue was removed, and circular templates of skin were generated using a 6 mm biopsy punch. A 3 mm biopsy punch was used to create an acute wound, and skin specimens were maintained at the air-liquid interface with DMEM (BioWhittaker), antibiotics-antimycotics (Invitrogen) and fetal bovine serum (FBS) (Gemimi Bio-Products) at 0 hours, 96 hours and 7 days. Acute wounds were topically treated at the time of wounding with 5 μM mimic miR-21 and miR-130a (Dharmacon) dissolved in 30% Pluronic F-127 (Sigma) gel in the presence of RNase inhibitor (Invitrogen). Fluorescent Cy3-labeled Pre-miR negative control (5 μM; Ambion) was used to follow the penetration into the epidermis of human skin explants. Frozen sections (7 μm) of acute wounds were stained with hematoxylin-eosin to follow the rate of healing. A Nikon Eclipse E800 microscope was used for visualization, and digital images were collected using SPOT Camera Advanced program.

Results:

Human skin explants are a useful model to study wound healing (Tomic-Canic, et al., Wound Repair Regen., 15(1):71-9 (2007)). Explanted human skin can be maintained at the air-liquid interface for 7-10 days and has the ability to heal 3 mm punch wounds during this time. To functionally evaluate specific miRNAs in human skin, the oligodeoxynucleotides method (Mori, et al., J. Exp. Med., 205(1):43-51 (2008)) was adapted to introduce miR-21 and miR-130a to an organ culture wound model. A dye-conjugated mimic and topical application was first used to document efficient incorporation in epidermis after wounding. Using this approach, it was shown that Cy3-conjugated mimic successfully penetrated into the epidermis of air-liquid maintained skin after 24 hours, and was still present in the epidermis of skin explants after 7 days. Therefore, it was shown that human skin organ culture can be used as a successful wound model for the further evaluation of the effects of specific miRNAs on wound healing. Next, mimic miR-21 and miR-130a were applied and the rate of healing as an outcome was observed. Four days after application, mimic mir-21 and miR-130a, but not mimic negative control or pluronic gel, markedly inhibited healing. The data collectively indicate that miR-21 and miR-130a have the ability to turn acute wounds into chronic non-healing wounds.

### Example 4

### TGFβRII and LepR are Downregulated in Epidermis of Chronic VUs

Materials and Methods:

Quantitative RT-PCR

RNA isolation and purification was performed using miRVana RNA isolation Kit (Ambion/Applied Biosystems, Austin, Tex.). For quantitative real-time PCR, 0.5 μg of total RNA from healthy skin and chronic wounds was reverse transcribed using a Omniscript Reverse Transcription kit (Qiagen). Real-time PCR was performed in triplicate using the Opticon2 thermal cycler (Bio-Rad, Hercules, Calif.) and detection system and an iQ SYBR Green Supermix (Bio-Rad, Hercules, Calif.). Relative expression was normalized for levels of HPRT1. The primer sequences used were:

HPRT1, forward
(5′-AAAGGACCCCACGAAGTGTT-3′, SEQ ID NO: 13)
and
reverse primer
(5′-TCAAGGGCATATCCTACAACAA-3′, SEQ ID NO: 14);
Smad7, forward
(5′-ACTCCAGATACCCGATGGATTT-3′, SEQ ID NO: 15)
and
reverse primer
(5′-CCTCCCAGTATGCCACCAC-3′, SEQ ID NO: 16);
TGFβRI forward
(5′-ACGGCGTTACAGTGTTTCTG-3′, SEQ ID NO: 17)
and
reverse primer
(5′-GCACATACAAACGGCCTATCT-3′, SEQ ID NO: 18);
TGFβII, forward
(5′-CCAAGGGCAACCTACAGGAG-3′, SEQ ID NO: 19)
and
reverse primer
(5′-GTGGAGGTGAGCAATCCCA-3′, SEQ ID NO: 20);
TGFβRIII forward
(5′-ACCTGTCAGTGCCTCCCAT-3′, SEQ ID NO: 21)
and
reverse primer
(5′-GAGCAGGAACACAACAGACTT-3′, SEQ ID NO: 22);

Statistical comparisons of expression levels from chronic wound vs healthy skin were performed using Student's t-test.

Immunohistochemistry

Paraffin sections were used for staining with anti-LepR antibody (Santa Cruz). Samples were deparaffinized and rehydrated followed by antigen retrieval. Retrieval was performed using DAKO at 90° F. for 30 minutes followed by a 20 minute cooling at room temperature. Endogenous peroxidase activity was quenched using 3% H₂O₂. The remainder of the protocol follows instructions in Vectastain Universal Kit. For visualization, DAB tablets were used. The samples were counterstained with Hematoxylin. As a negative control, 1×PBS was substituted for primary antibody. Finally the samples were dehydrated and mounted using permanent mounting media. For visualization, a Nikon Eclipse E800 microscope was used and digital images were collected using SPOT Camera Advanced program.

Results:

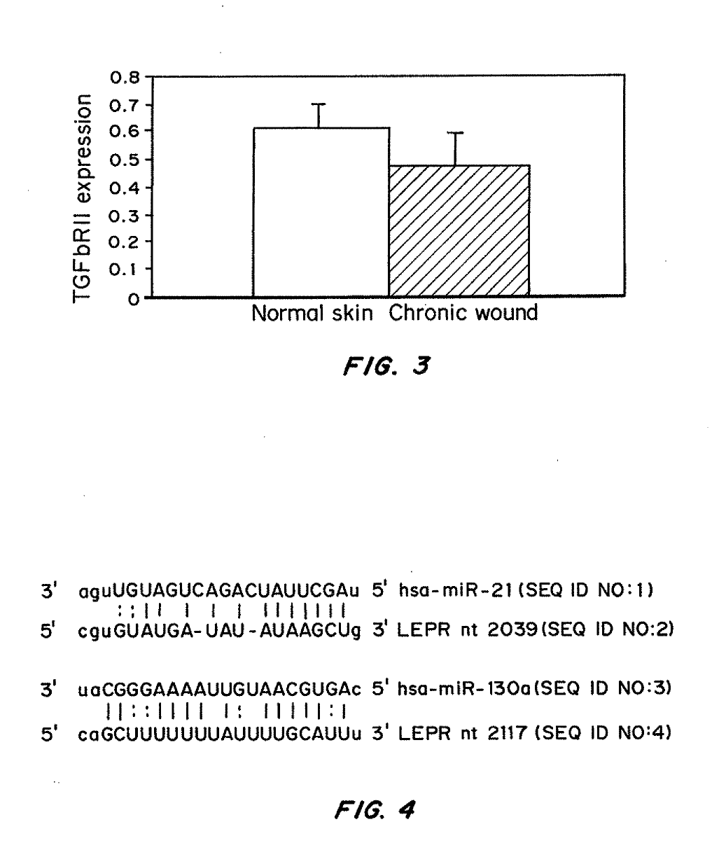

Because a single miRNA molecule targets a large subset of mRNAs, it is important to verify its downstream targets in order to validate that induction of a particular miRNA contributes to pathogenesis. Thus, patient biopsies were used to validate downstream targets of miRNAs identified as induced in venous ulcers. If induction of miRNA leads to down-regulation of target mRNA, it was expected to find either mRNA or protein down-regulation of target gene expression after treatment with miRNA. TGFβRII and LepR are known to be downstream targets of several of the miRNAs that were up-regulated in venous ulcers. Quantitative RT-PCR was used to measure mRNA expression levels of TGFβRII, RII and RIII in cell explants that had been treated with miRNA-21 (FIG. 3). It was found that the expression levels of TGFβRII mRNA were not affected in VUs, whereas TGFβRI and TGFβRIII mRNA levels were lower in non-healing edges of VUs than in control skin. However, immunohistochemistry revealed profound down-regulation of both TGFβRI and TGFβRII protein in VUs in comparison to control skin. These data suggest that TGFβRII is suppressed at a post-translational translational level by miRNAs. Indeed, TGFβRII has been verified as a target for miR-20a and miR-21 (Volinia, et al., Proc. Natl. Acad. Sci. 103(7):2257-61 (2006)), and both of these miRNAs were up-regulated in venous and diabetic foot ulcers.

Next, the expression of the leptin receptor, which is important for wound healing, was examined. Leptin is a hypoxia-inducible pleiotropic cytokine known to participate in multiple cellular and physiological processes. Genetic defects in leptin production or leptin receptor expression in mice results in a complex phenotype with multiple metabolic and reproductive alterations that include development of morbid obesity, metabolic syndrome, and infertility. The ob/ob (leptin null), and db/db (leptin receptor null), mouse strains are characterized by severe impairment in their ability to repair cutaneous wounds and have been extensively used as models of pathological wound healing (Greenhalgh, et al., Am. J. Pathol., 136(6):1235-46 (1990); Tsuboi, et al., J. Dermatol., 19(11):673-5 (1992); Brem, et al., Exp. Gerontol., 42(6):523-31 (2007)). Systemically and topically supplemented leptin improves re-epithelialization of wounds in ob/ob animals (Frank, et al., J. Clin. Invest, 106(4):501-9 (2000)). The expression of LepR in chronic venous ulcers was not previously analyzed. A suppression of LepR expression was observed in all tested chronic VUs, specifically in a basal layer of epidermis. However, the expression of LepR was induced in keratinocytes located at the wound margin during acute healing, as has been reported previously (Frank, et al., J. Clin. Invest., 106(4):501-9 (2000)).

### Example 5

### miR21 and miR-130a Target 3′UTR of LepR

Materials and Methods:

Luciferase Assays

For dual-luciferase assays, 1 μg pGL3 reporter, 5 μg pSilencer constructs and 100 ng Renilla luciferase control (Promega) were transfected with Fugene6 reagent (Roche) into primary human keratinocytes. Plasmid containing sense LepR 3′ UTR sequence was used as a reporter (pGL3-LepR 3′S), whereas the plasmid containing the 3′ UTR sequence in the antisense orientation was used as a negative control (pGL3-LepR 3′AS). The relative luciferase activities were determined using the Dual-Glo Luciferase Assay system (Promega) 48 h after transfection

Results:

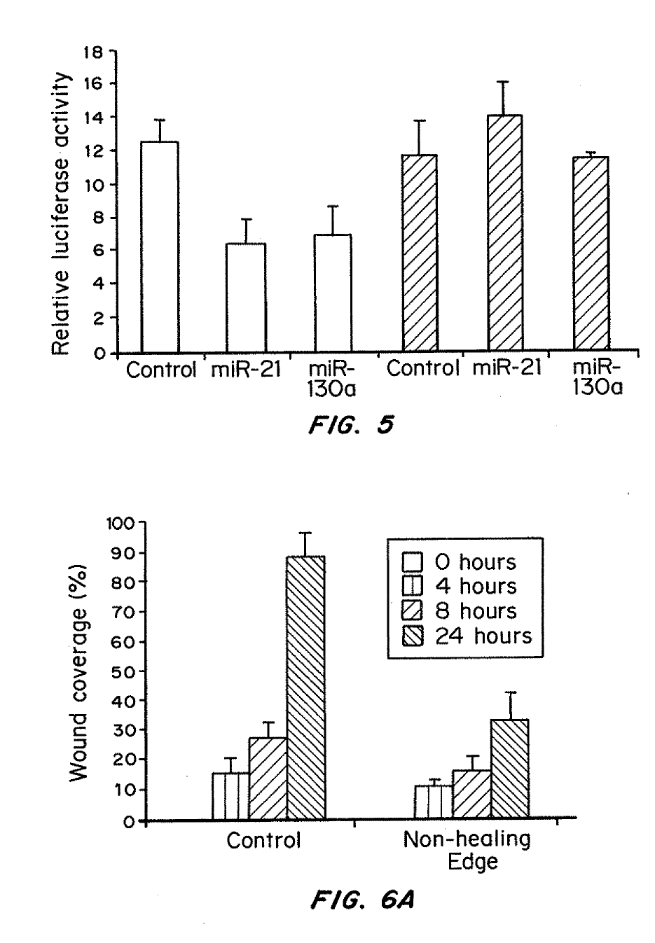

Heptamer and hexamer sequences that are complementary to miR-21 and miR-130a 5′ seed sequences, respectively are found within the 3′ UTR of LepR mRNA (FIG. 4). When introduced into the 3′UTR of a luciferase reporter gene, a 2259-base-pair human LepR 3′UTR fragment encompassing these two putative target sites caused a significant reduction in activity in miR-21- and miR-130a-expressing keratinocytes (FIG. 5). miR-21 and miR130a did not significantly inhibit luciferase activity in the two control constructs, namely, control without 3′ UTR or that with the LepR 3′ UTR in the antisense (AS) orientation (FIG. 5). Together, these results indicate that miR-21 and miR-130a have a direct effect on LepR mediated through 3′UTR target sites.

### Example 6

### Development of an In Vitro Model for Validating Candidate Treatments and Targets

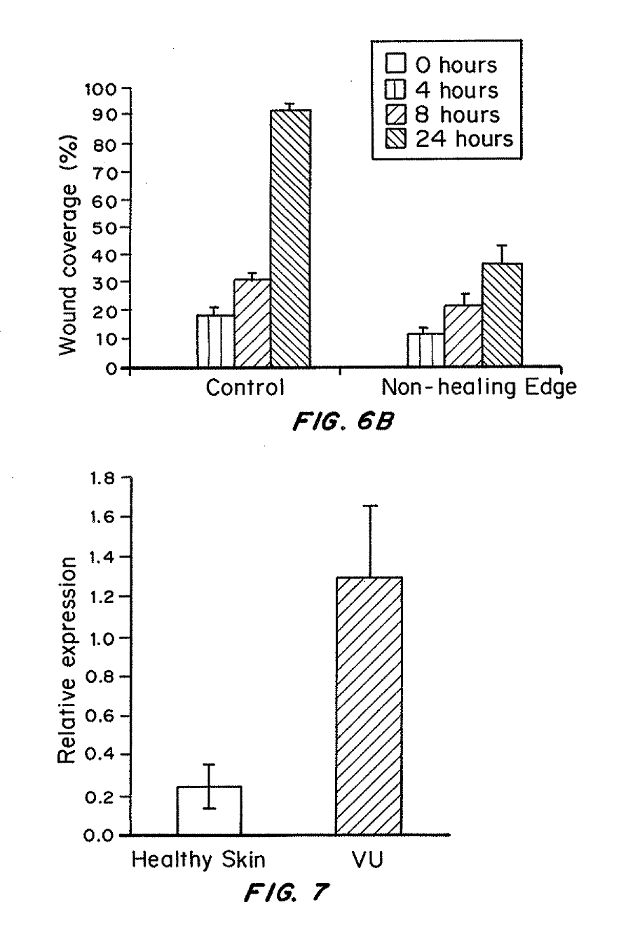

To develop an in vitro model for studies of the cellular and molecular mechanisms of wound healing, methods for establishing primary cultures of fibroblasts isolated from patient wounds were developed (Brem, et al., J. Transl. Med, 6:75 (2008)). Routine surgical debridement to remove the rim of necrotic infected tissue from the edge of the wound and leave a clean edge of healing-competent tissue is part of the standard of care for chronic DFUs at New York University. Fibroblasts cultured from chronic wound sites maintain their pathogenic phenotype (Brem, et al., Mol. Med., 13(1-2):30-9 (2007)): cells from the non-healing edge are misshaped, inflated with enlarged nuclei and clumped together compared to control fibroblasts. To functionally evaluate the non-healing phenotype of these primary cells an in vitro wound scratch assay was used to quantitatively measure cell migration. When normal human fibroblasts grown in a tissue culture dish are “wounded” by a scratch, they migrate over the scratch to close the gap. Cultured primary cells originating from non-healing edge of a chronic ulcer have markedly reduced migration capacity (FIG. 6A). Furthermore, when incubated in the presence of growth factors that stimulate wound healing in vivo, such as VEGF or GM-CSF, primary cells from non-healing edge do not respond to growth factor stimuli (FIG. 68). A distinct response correlating to each wound location was found. Specifically, fibroblasts grown from the non-healing edges of chronic ulcers have the slowest migration rate, covering only 33% of the initial scratch in 24 hours, whereas fibroblasts from the adjacent non-ulcerated area (obtained post-debridement) covered 75% of the initial scratch and were only slightly slower than control, which closed 89% of scratched area. These data demonstrate that primary cells in culture maintain functional characteristics of the non-healing wound and can be utilized for testing of potential therapy.

### Example 7

### miRNA Upregulation in Venus Ulcers

miRNA expression was investigated in venus ulcers using in situ hybridization with miR-21, miR-130a, and miR-203 LNA probes. miR-21, miR-130a, and miR-203 were all induced in the epidermis of venus ulcers. Moreover, miR-21 and miR-130a were also upregulated in dermal fibroblasts and endothelial cells.

The relative expression of Dicer was investigated to explain the induction of multiple miRNA molecules simultaneously in venus ulcers. As shown in FIG. 7, Dicer is upregulated in venus ulcers in venus ulcers compared to healthy skin. This may explain the mechanism by which the miRNA molecules are induced in wounds such as venus ulcers.

### Example 8

### Effect of miR21 on Wound Healing

To investigate the role of miR-21 on wound healing, miR-21 and an antagomir that inhibits miR-21 were administered to rat acute wounds. miR-21 inhibited epithelialization of the wound. In contrast, local delivery of antogomir21 promoted epithelialization in the acute wounds in vivo 5 days post wounding compared to untreated control wounds. The antagomir treatment not only fully closed the wound but also enhanced the epithelial thickness.

### Example 9

### miRNA Upregulation in Pseudomonas Wounds

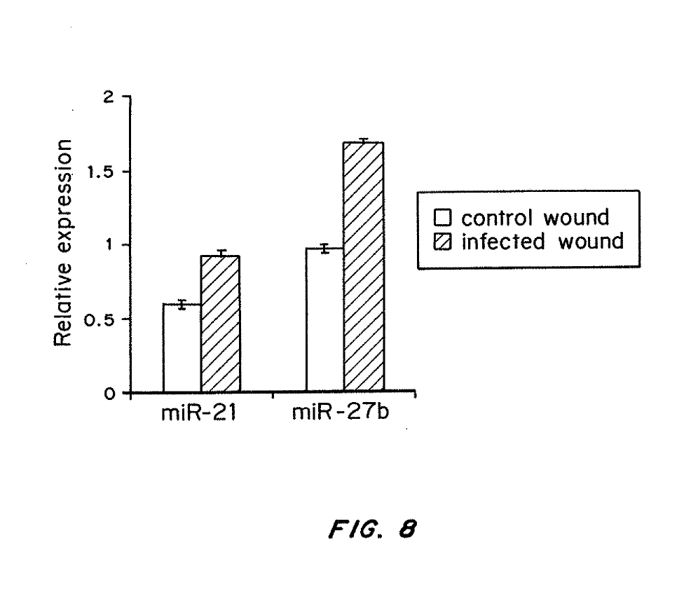

miRNA expression of miR-21 and miR-27b was investigated in an in vivo porcine wound model infected with biofilm-forming Pseudomonas. As shown in FIG. 8, both miR-21 and miR-27b were upregulated in the infected wounds.

We claim:

1. A method for treating an inflammatory condition comprising administering to a subject in need thereof a pharmaceutical composition comprising
a microRNA (miRNA) antagonist in an effective amount to reduce one or more symptoms of an inflammatory disorder and optionally, and a pharmaceutical excipient,
wherein the miRNA antagonist reduces the amount of, or inhibits the biological function of, an miRNA or pre-miRNA that is upregulated in epithelial cells at a non-healing edge of a chronic, non-healing wound as compared to normal epithelial cells.

2. The method of claim 1, wherein the miRNA antagonist is administered in an effective amount to promote wound healing.

3. The method of claim 2, wherein the wound is a chronic, non-healing wound.

4. The method of claim 3, wherein the chronic, non-healing wound is selected from the group consisting of diabetic ulcers, arterial ulcers, venous ulcers, pressure ulcers, mouth ulcers, sickle cell ulcers, corticosteroid-induced wounds and burns.

5. The method of claim 4, wherein the diabetic ulcer is a diabetic foot ulcer.

6. The method of claim 2, wherein the wound is an acute wound caused by injury or surgery.

7. The method of claim 2, wherein the wound is in a tissue selected from the group consisting of skin, mouth tissue, gingiva, and corneal epithelium.

8. The method of claim 2, wherein the miRNA antagonist comprises an oligonucleotide that binds to and inhibits or reduces the expression of an miRNA or pre-miRNA that is upregulated in epithelial cells at a non-healing edge of a chronic, non-healing wound as compared to normal epithelial cells.

9. The method of claim 8, wherein the miRNA or pre-miRNA is miR-21.

10. The method of claim 8, wherein the miRNA or pre-miRNA is selected from the group consisting of miR-590-5p, miR-15a, miR-15b, miR-16-1, miR-16-2, miR-195, miR-424, miR-497, miR-17-5p, miR-20a, miR-20b, miR-93.mr, miR-106a, miR-106b, miR-519.d, miR-106a, miR-106b, miR-302a, miR-302b, miR-302c, miR-302d, miR-302e, miR-302f, miR-103-1, miR-103-2, miR-103-1-as, miR-103-2-as, miR-107, miR-130a, miR-130b, miR-301a, miR-301b, miR-27a, miR-27b, miR-143, miR-146a, miR-146b and miR-203.

11. The method of claim 8, wherein the oligonucleotide is an antagomir comprising a phosphorothioate backbone, 2′-O-methylation of sugars and a terminal cholesterol moiety.

12. The method of claim 2, wherein the pharmaceutical composition is administered topically or subcutaneously.

13. A pharmaceutical composition for promoting wound healing comprising an effective amount of microRNA (miRNA) antagonists to promote wound healing in a formulation for topical or subcutaneous administration,
wherein the miRNA antagonist reduces the amount of, or inhibits the biological activity of, a microRNA (miRNA) that is upregulated in epithelial cells at a non-healing edge of a chronic, non-healing wound as compared to normal epithelial cells.

14. The pharmaceutical composition of claim 13, wherein the miRNA or pre-miRNA is human miR-21.

15. The pharmaceutical composition of claim 13, wherein the miRNA or pre-miRNA is selected from the group consisting of human miR-590-5p, miR-15a, miR-15b, miR-16-1, miR-16-2, miR-195, miR-424, miR-497, miR-17-5p, miR-20a, miR-20b, miR-93.mr, miR-106a, miR-106b, miR-519.d, miR-106a, miR-106b, miR-302a, miR-302b, miR-302c, miR-302d, miR-302e, miR-302f, miR-103-1, miR-103-2, miR-103-1-as, miR-103-2-as, miR-107, miR-130a, miR-130b, miR-301a, miR-301b, miR-27a, miR-27b, miR-143, miR-146a, miR-146b and miR-203.

16. The pharmaceutical composition of claim 13, wherein the miRNA antagonist comprises an oligonucleotide that binds to and inhibits or reduces the expression of an miRNA or pre-miRNA that is upregulated in epithelial cells at a non-healing edge of a chronic, non-healing wound as compared to normal epithelial cells.

17. The pharmaceutical composition of claim 16, wherein the oligonucleotide comprises one or more modified bases, modified sugar groups, modified phosphate groups, modified nucleoside linkages, terminal modifications, or combinations thereof.

18. The pharmaceutical composition of claim 16, wherein the oligonucleotide is an antagomir comprising a phosphorothioate backbone, 2′-O-methylation of sugars and a terminal cholesterol moiety.

19. The pharmaceutical composition of claim 13 wherein the pharmaceutical composition is formulated for controlled or sustained release.

20. The pharmaceutical composition of claim 13, further comprising additional active agents selected from the group consisting of anti-microbial agents, pain relievers, anti-inflammatory agents, growth factors, and vitamins.


SEQUENCE LISTING (22 sequences)

SEQ ID NO: 1 - RNA, 22 nt, Homo sapiens
uagcuuaucagacugauguuga

SEQ ID NO: 2 - RNA, 20 nt, Homo sapiens
cguguaugauauauaagcug

SEQ ID NO: 3 - RNA, 22 nt, Homo sapiens
cagugcaauguuaaaagggcau

SEQ ID NO: 4 - RNA, 22 nt, Homo sapiens
cagcuuuuuuuauuuugcauuu

SEQ ID NO: 5 - DNA, 29 bp, Artificial Sequence
  Synthetic Construct
cgggatccttatcaaatcctgcctgactg

SEQ ID NO: 6 - DNA, 32 bp, Artificial Sequence
  Synthetic Construct
cccaagcttgaccagagtttctgattataaca

SEQ ID NO: 7 - DNA, 29 bp, Artificial Sequence
  Synthetic Construct
cgggatccgctgtattgaagcaaagaagg

SEQ ID NO: 8 - DNA, 27 bp, Artificial Sequence
  Synthetic Construct
cccaagcttgggtagctgactggtgcc

SEQ ID NO: 9 - DNA, 33 bp, Artificial Sequence
  Synthetic Construct
gctctagaagtctaatcatgatcactacagatg

SEQ ID NO: 10 - DNA, 31 bp, Artificial Sequence
  Synthetic Construct
gctctagagaaaaatcctgccaaacaactac

SEQ ID NO: 11 - DNA, 36 bp, Artificial Sequence
  Synthetic Construct
gctctagatttcactgaagaaaccttcagatttgtg

SEQ ID NO: 12 - DNA, 35 bp, Artificial Sequence
  Synthetic Construct
gctctagaggatttaaatctgaactctctgattcc

SEQ ID NO: 13 - DNA, 20 bp, Artificial Sequence
  Synthetic Construct
aaaggaccccacgaagtgtt

SEQ ID NO: 14 - DNA, 22 bp, Artificial Sequence
  Synthetic Construct
tcaagggcatatcctacaacaa

SEQ ID NO: 15 - DNA, 22 bp, Artificial Sequence
  Synthetic Construct
actccagatacccgatggattt

SEQ ID NO: 16 - DNA, 19 bp, Artificial Sequence
  Synthetic Construct
cctcccagtatgccaccac

SEQ ID NO: 17 - DNA, 20 bp, Artificial Sequence
  Synthetic Construct
acggcgttacagtgtttctg

SEQ ID NO: 18 - DNA, 21 bp, Artificial Sequence
  Synthetic Construct
gcacatacaaacggcctatct

SEQ ID NO: 19 - DNA, 20 bp, Artificial Sequence
  Synthetic Construct
ccaagggcaacctacaggag

SEQ ID NO: 20 - DNA, 19 bp, Artificial Sequence
  Synthetic Construct
gtggaggtgagcaatccca

SEQ ID NO: 21 - DNA, 19 bp, Artificial Sequence
  Synthetic Construct
acctgtcagtgcctcccat

SEQ ID NO: 22 - DNA, 21 bp, Artificial Sequence
  Synthetic Construct
gagcaggaacacaacagactt